(19) United States
(12) Patent Application Publication
(10) Pub. No.: US 2012/0258482 A1
(43) Pub. Date: Oct. 11, 2012

(54) PCNA METHYLTRANSFERASE

(75) Inventors: Derek J. Hoelz, Bangor, ME (US); Robert J. Hickey, Indianapolis, IN (US); Linda H. Malkas, Indianapolis, IN (US)

(73) Assignee: INDIANA UNIVERSITY RESEARCH AND TECHNOLOGY CORPORATION, INDIANAPOLIS, IN (US)

(21) Appl. No.: 13/500,587

(22) PCT Filed: Oct. 7, 2010
(86) PCT No.: PCT/US10/51839

§ 371 Date: Jun. 28, 2012

Related U.S. Application Data
(60) Provisional application No. 61/250,271, filed on Oct. 9, 2009.

(51) Int. Cl. C12Q 1/48; G01N 27/26; G01N 27/62; C12N 9/10

(52) U.S. Cl. 435/15; 435/193

(57) ABSTRACT

Proliferating cell nuclear antigen (PCNA)-dependent glutamate methyltransferases are disclosed that can methylesterify one or more glutamic acid or aspartic acid residues of PCNA.

### CROSS REFERENCE TO RELATED APPLICATIONS

This application claims priority to U.S. Provisional Patent Application No. 61/250,271, filed on Oct. 09, 2009 the disclosure of which is hereby expressly incorporated by reference in its entirety.

### INCORPORATION OF SEQUENCE LISTING

The sequence listing with file name 213943_SL.txt, created on Oct. 7, 2010 (37.8 KB) is expressly incorporated by reference in its entirety.

### FIELD

The present disclosure relates to identification and isolation of methyltransferases capable of methylesterifying one or more acidic residues of PCNA.

### BACKGROUND

One of the least understood and most complex disease processes is the transformation that occurs as a cell becomes malignant. This process involves both genetic mutations and proteomic transformations, the result of which, allows the cell to escape normal controls that prevent inappropriate cell division. Cancer cells share some common attributes. Most cancer cells proliferate outside of the normal cell cycle controls, exhibit morphological changes and exhibit various biochemical disruptions to cellular processes.

Cancer is usually diagnosed when a tumor becomes visible well after the first on-set of cellular changes. Many cancers are diagnosed after a biopsy sample is examined by histology for morphologic abnormalities, evidence of cell proliferation and genetic irregularities. Effective treatment for malignancy often depends on the ability to detect reliably, the presence of malignant cells at early stages of a disease so that an effective treatment can begin at a stage when the disease is more susceptible to such treatment. Thus, there is a need to be able to reliably detect a potentially malignant cell that has not progressed to the histological stage recognized as malignant, but which can progress to a malignant state. There is also a need for a rapid, minimally invasive technique that can reliably detect malignant cells or potentially malignant cells.

Proliferating cell nuclear antigen (PCNA) is a 29 kDa nuclear protein and its expression in cells during the S and G2 phases of the cell cycle, makes the protein a good cell proliferation marker. It has also been shown to partner in many of the molecular pathways responsible for the life and death of the cell. Its periodic appearance in S phase nuclei suggested an involvement in DNA replication. PCNA was later identified as a DNA polymerase accessory factor in mammalian cells and an essential factor for SV40 DNA replication in vitro. In addition to functioning as a DNA sliding clamp protein and a DNA polymerase accessory factor in mammalian cells, PCNA interacts with a number of other proteins involved in transcription, cell cycle checkpoints, chromatin remodeling, recombination, apoptosis, and other forms of DNA repair. Besides being diverse in action, PCNA's many binding partners are linked by their contributions to the precise inheritance of cellular functions by each new generation of cells. PCNA may act as a master molecule that coordinates chromosome processing.

PCNA is also known to interact with other factors like FEN-1, DNA ligase, and DNA methyl transferase. Additionally, PCNA was also shown to be an essential player in multiple DNA repair pathways. Interactions with proteins like the mismatch recognition protein, Msh2, and the nucleotide excision repair endonuclease, XPG, have implicated PCNA in processes distinct from DNA synthesis. Interactions with multiple partners generally rely on mechanisms that enable PCNA to selectively interact in an ordered and energetically favorable way.

Clues to a mechanism of PCNA's functions were initially uncovered through investigation of the DNA synthesome, a multiprotein DNA replication complex present in mammalian cells. Studies examining the synthetic activity of the DNA synthesome identified an increased error rate in malignant cells when compared to non-malignant cells. These results suggest that a structural alteration to one or more components of the DNA synthesome in malignant cells has occurred. 2D-PAGE immunoblot analysis of PCNA, an essential component of the DNA synthesome, revealed two distinct isoforms with vastly different isoelectric points (pIs). One PCNA isoform displayed a significantly basic pI and was present in both malignant and non-malignant cells. The other isoform had an acidic pI and was found exclusively in malignant cells. Because of its presence only in malignant cells, this isoform was termed the cancer-specific isoform or caPCNA, and the post-translational alteration that is responsible for PCNA's altered 2D-PAGE migration pattern remains undetermined.

Some labeling studies with PCNA suggested that the migration of PCNA was most likely not due to alterations such as phosphorylation, acetylation, glycosylation, or sialyzation. Conflicting studies have surfaced attempting to identify post-translational modifications to PCNA. For example, the phosphorylation of PCNA was reported to affect its binding to sites of DNA synthesis. Another study claimed that PCNA was, after all, not phosphorylated but acetylated. In addition to these studies, analysis of yeast PCNA has shown it to be the target of ubiquitination in response to DNA damage and sumoylation in the absence of damage. Due to the diverse and conflicting structural evidence for PCNA, it is difficult to identify which modifications, if any, are responsible for the appearance and functions of caPCNA isoform.

Therefore, identification of the correct post-translational modifications of caPCNA is desirable to develop diagnostic methods and also to develop therapeutics based on the interactions of caPCNA with its partners. Malignant cancer cells express an isoform of PCNA termed cancer specific PCNA (caPCNA) and non-malignant cells express an isoform termed non-malignant PCNA (nmPCNA). Effective compositions and methods to distinguish the two isoforms are needed for diagnosis and treatment of cancers.

Methyl esterification of glutamic acid residues in proteins has only been observed in chemotactic bacteria where a specialized glutamate carboxyl O-methyltransferase, CheR, modifies specific residues of the chemotaxis receptor during signal transduction. In eukaryotic cells, however, only three carboxyl O-methyltransferases are known to exist and they do not have specificity for glutamic acid residues.

A glutamate O-methyltransferase (EC 2.1.1.80) is an enzyme that catalyzes a chemical reaction represented by a general equation: S-adenosyl-L-methionine+protein L-glutamate→S-adenosyl-L-homocysteine+protein L-glutamate methyl ester. S-adenosyl methionine and the glutamic acid of the target protein are the substrates of the enzyme.

### SUMMARY

One embodiment of the present disclosure is directed to the isolation and identification of a proliferating cell nuclear antigen (PCNA)-dependent glutamate carboxyl O-methyltransferase. More particularly, in one embodiment the methyltransferease is a human methyltransferase that methylesterifies one or more acidic amino acid residues of proliferating cell nuclear antigen (PCNA). Sequence alignments with other protein carboxyl O-methyltransferases demonstrated the existence of consensus and conserved regions within this protein, and secondary structural predictions confirm its potential to form a SAM-dependent methyltransferase fold in its C-terminus.

In accordance with one embodiment a purified methyltransferase and derivatives thereof are provided, where the methyltransferase methylesterifies one or more acidic amino acid residues of proliferating cell nuclear antigen (PCNA) and has a molecular weight of approximately 50 kDa. In a further embodiment the methyltransferase has a secondary structure comprising 9 α-helices in its N-terminus and seven a-helices and nine β-sheets in its C-terminus. In one embodiment the methyltransferase methylesterifies one or more glutamic acid or aspartic acid residues that correspond to the amino acid positions 3, 85, 93, 94, 104, 109, 115, 120, 132, 143, 174, 189, 201, 238, 256, and 258 of the peptide of SEQ ID NO: 37.

In one embodiment a purified methyltransferase is provided comprising the sequence of SEQ ID NO: 33 or an amino acid sequence that is greater than 75%, 80%, 85%, 90%, 95% of 99% identical to the corresponding sequence of SEQ ID NO: 33 and has activity for methylesterifying one or more acidic amino acid residues of PCNA. In accordance with one embodiment the methyltransferase comprises an amino acid sequence of SEQ ID NO: 33 wherein 1-20, 1-15, 1-10, 1-5 or 1-3 amino acids have been modified relative to the amino acid sequence of SEQ ID NO: 33. In one embodiment those modification comprise conservative amino acid substitutions. In accordance with one embodiment the methyltransferease comprises an amino acid derivative of SEQ ID NO: 33 wherein the peptide is modified in a nonconservative region of the peptide. For example, in one embodiment the modifications do not impact amino acids 245-257 of the methyltransferase of SEQ ID NO: 1, or other conserved regions.

The unique methyltransferase of SEQ ID NO: 33 is involved in DNA replication and repair. As disclosed herein the activity of the enzyme is also correlated with cancer and decreased activity of the enzyme can be used as a diagnostic marker for cancer, including breast cancer. In accordance with one embodiment a method for diagnosing the presence of cancer or a pre-cancerous condition is provided. The method comprises determining the level of activity of the proliferating cell nuclear antigen (PCNA)-dependent glutamate carboxyl O-methyltransferase and determining if that activity falls below a threshold level to indicate a risk of cancer.

In one embodiment a kit is provided for conducting methylesterification assays to measure the level of methyltransferase activity in a sample. In one embodiment the kit comprises the novel proliferating cell nuclear antigen (PCNA)-dependent glutamate carboxyl O-methyltransferase disclosed herein and proliferating cell nuclear antigen (PCNA) polypeptide of SEQ ID NO: 37 or a peptide comprising an amino acid sequence selected from the group consisting of

(SEQ ID NO: 16)
MFEAR;
(SEQ ID NO: 17)
IEDEEGS;
(SEQ ID NO: 18)
IEDEEGS;
(SEQ ID NO: 19)
VSDYEMK;
(SEQ ID NO: 20)
MPSGEFAR;
(SEQ ID NO: 21)
LSQTSNVDK;
(SEQ ID NO: 22)
CAGNEDIITLR;
(SEQ ID NO: 23)
FSASGELGNGNIK;
(SEQ ID NO: 24)
AEDNADTLALVFEAPNQEK;
(SEQ ID NO: 25)
AEDNADTLALVFEAPNQEK;
(SEQ ID NO: 26)
AEDNADTLALVFEAPNQEK;
(SEQ ID NO: 27)
AEDNADTLALVFEAPNQEK;
(SEQ ID NO: 28)
LMDLDVEQLGIPEQEYSCVVK;
(SEQ ID NO: 29)
ATPLSSTVTLSMSADVPLVVEYK;
(SEQ ID NO: 30)
LSQTSNVDKEEEAVTIEMNEPVQLTFALR;
and
(SEQ ID NO: 32)
LMDLDVEQLGIPEQEYSCVVK.

In one embodiment the kit further comprises S-adenosyl-L-methionine.

In one embodiment the present invention is directed to nucleic acid sequences that encode the methyltransferase of SEQ ID NO: 33, and in one embodiment the nucleic acid sequence comprises the sequence of SEQ ID NO: 36. The nucleic acid sequence encoding the proliferating cell nuclear antigen (PCNA)-dependent glutamate carboxyl O-methyltransferase may comprise part of a larger nucleic acid construct including an expression vector. In accordance with one embodiment the vector is a eukaryotic expression vector. Host cells comprising the methyltransferase nucleic acid sequences are also encompassed by the present invention.

Additional features of the present disclosure will become apparent to those skilled in the art upon consideration of the following detailed description of embodiments exemplifying the best mode of carrying out the subject matter of the disclosure as presently perceived.

### BRIEF DESCRIPTION OF THE DRAWINGS

FIG. 1 shows a schematic illustration of the development of a PCNA-dependent carboxyl methyl transferase assay (“vapor diffusion assay”).

FIGS. 2A & 2B are bar graphs presenting data produced from in vitro cell extract assays demonstrating that PCNA is the target of a carboxyl methyltransferase in human cells. SAM-dependent carboxyl methyltransferase activity was measured in MCF7 cell extracts using the vapor diffusion assay. FIG. 2A shows PCNA-dependent carboxyl methyltransferase activity is present in the cell extracts and FIG. 2B demonstrates that the carboxyl methyl transferase activity detected in MCF7 cells is enzymatic and specific for PCNA. Average activities from representative assays are presented ±S.E.M. Extracts were denatured by heating at 95° C. for 5 min. Results were assessed using a two-tailed t-test and activities significantly different (P<0.05) from MCF7 control extracts are denoted with an asterisk. FIG. 2B legend: A=no PCNA; B=2 μg rPCNA; C=5 μg rPCNA; D=10 μg rPCNA; E=2 μg BSA; F=5 μg BSA; G=10 μg BSA; H=no lysate; I=Inactivated (boiled) lystae; J=Inactivated 2 μg rPCNA.

FIG. 3 shows identification of a PCNA-dependent carboxyl methyltransferase—ARM1. FIG. 3A. Cell extracts were equilibrated with 30% NH₄SO₄ and the soluble fraction loaded onto a phenyl Sepharose column. Fractions were assayed for PCNA-dependent methyltransferase activity and the phenyl sepharose chromatogram shows enrichment of activity. FIG. 3B is a drawing of a hypothetical SAM-dependent methyltransferase secondary structure proposed for the PCNA-dependent carboxyl methyltransferase. Arrows denote the location of conserved SAM-dependent binding motifs and consensus regions.

FIG. 4 shows an alignment of the E. coli glutamyl methyltransferase CheR (top sequence; SEQ ID NO: 34), the hypothetical protein sequence of the C6orf211 methyltransferase (middle sequence; SEQ ID NO: 33) and the human protein repair enzyme protein iso-aspartate methyltransferase (PIMT; bottom sequence, SEQ ID NO: 35)

FIGS. 5A-5D Extended sequences containing CheR motifs I and II and region III were aligned to the full-length C6orf211 protein sequence. Conserved glycine and glutamic acid residues in CheR motif I and catalytic aspartic acid and conserved isoleucine residues of CheR motif II are underlined and homologous sequences boxed. Full-length CheR and C6orf211 protein sequences were submitted to Nomad and the aligned sequence is shown with CheR region II underlined (FIG. 5A). The relative positions of motifs I, II and regions II and III within CheR and the C6orf211 protein are shown in FIG. 5B. A Flag-Arm1 (C6orf211 product) was transiently expressed in breast cancer cells and immunoprecipitated with anti-Flag antibodies. FIG. 5C represents Western analysis of whole cell extracts (WCE) and immunoprecipitated fractions from control and Flag-Arm1 expressing cells showing efficient isolation of Flag-Arm1. Immunoprecipitated fractions from control and Flag-Arm1 expressing cells were assayed for carboxyl methyltransferase activity in the absence and presence of 2 μg of purified 6× His-PCNA, and PCNA-dependent carboxyl methyltransferase activity shown in FIG. 5D. Assays were performed in triplicate and shown ±S.E.M. Significant differences in activity were identified by two-tailed t-test, p<0.05.

FIG. 6 shows evolutionary alignments of the C6orf211 gene products from eight different eukaryotic species: H. sapiens (SEQ ID NO: 33); B. tarus (SEQ ID NO: 38); M. musculus (SEQ ID NO: 39); D. rerio (SEQ ID NO: 40); D. melanogaster (SEQ ID NO: 41); C. elgans (SEQ ID NO: 42); S. pombe (SEQ ID NO: 43); and S. cerevisia (SEQ ID NO: 44). The alignments reveal high conservation in this sequence and 100% conservation of the glutamic acid and glycine residues, suggesting that these residues are important for the protein's function.

FIG. 7 shows the results of experiments relating to p53- and p21-dependent methyl esterification of PCNA following genotoxic stress. FIG. 7A is a Western blot analysis of p53 wild-type (MCF7) and p53-mutant (MDA-MB-468) breast carcinoma cell lines exposed to 5 μM DOX for the indicated times. Cell extracts (200 μg) were resolved by 2D-PAGE and immunoblotted with anti-PCNA antibodies. In FIG. 7B extracts from MCF7 cells (50 μg) treated with 5 μM DOX were separated by 12% SDS-PAGE and immunoblotted with anti-p21WAF1 antibodies. FIG. 7C is a Western blot analysis of glutathione-immobilized GST-p21 and GST-PIP (a.a. 139-160) fusion proteins incubated with untreated MCF7 cell extracts for 2 h at 4° C. and the pull-down fractions were resolved by 2D-PAGE and Western blotted for PCNA. PCNA isoforms were isolated from MCF7 extracts by incubation with PIP-affinity beads for ˜2 h and analyzed by 2D-PAGE using pH 4-7 IPG strips. Colloidal Coomassie stained spots (See FIG. 7D) were excised from the gel, trypsin digested, and sequenced by LC-MS/MS.

FIG. 8 is a bar graph showing data from an experiment where methyltransferase activity was measured in cancer (MCF7) and non-cancerous (MCF 10A) cell lines. Lane 1 represents a control wherein the reaction was conducted in the absence of a cell extract (rPCNA); Lane 2 represents the reaction conducted using a cell extract from a malignant breast cell but in the absence of substrate (MCF7); Lane 3 represents the reaction conducted using a cell extract from a malignant breast cell plus substrate (MCF7+rPCNA) ; Lane 4 represents the reaction conducted using a cell extract from a non-malignant breast cell in the absence of substrate (MCF 10A); and Lane 5 represents the reaction conducted using a cell extract from a non-malignant breast cell plus substrate (lane 3; MCF 10A+rPCNA). The results show that non-malignant breast cells contain higher levels of MT activity.

FIG. 9 shows an amino acid sequence alignment of putative PCNA-dependent methyltransferase partial sequence ORF with known methyltransferase domains. The PCNA methyltransferase sequence aligns to the bacterial glutamate methyltransferase.

### DETAILED DESCRIPTION

Definitions

In describing and claiming the invention, the following terminology will be used in accordance with the definitions set forth below.

As used herein, the term “purified” and like terms relate to the isolation of a molecule or compound in a form that is substantially free of contaminants normally associated with the molecule or compound in a native or natural environment. As used herein, the term “purified” does not require absolute purity; rather, it is intended as a relative definition. The term “purified polypeptide” is used herein to describe a polypeptide which has been separated from other compounds including, but not limited to nucleic acid molecules, lipids and carbohydrates.

The term “isolated” requires that the referenced material be removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated.

As used herein, the term “peptide” encompasses a sequence of 3 or more amino acids and typically less than 50 amino acids, wherein the amino acids are naturally occurring or non-naturally occurring amino acids. Non-naturally occurring amino acids refer to amino acids that do not naturally occur in vivo but which, nevertheless, can be incorporated into the peptide structures described herein.

As used herein, the terms “polypeptide” and “protein” are terms that are used interchangeably to refer to a polymer of amino acids, without regard to the length of the polymer. Typically, polypeptides and proteins have a polymer length that is greater than that of “peptides.”

As used herein an amino acid “substitution” refers to the replacement of one amino acid residue by a different amino acid residue.

As used herein, the term “conservative amino acid substitution” is defined herein as exchanges within one of the following five groups:

I. Small aliphatic, nonpolar or slightly polar residues:
Ala, Ser, Thr, Pro, Gly;

II. Polar, negatively charged residues and their amides and esters:
Asp, Asn, Glu, Gln, cysteic acid and homocysteic acid;

III. Polar, positively charged residues:
His, Arg, Lys; Ornithine (Orn)

IV. Large, aliphatic, nonpolar residues:
Met, Leu, Ile, Val, Cys, Norleucine (Nle), homocysteine

V. Large, aromatic residues:
Phe, Tyr, Trp, acetyl phenylalanine

As used herein an amino acid “modification” refers to a substitution, addition or deletion of an amino acid, and includes substitution with or addition of any of the 20 amino acids commonly found in human proteins, as well as atypical or non-naturally occurring amino acids.

The term “identity” as used herein relates to the similarity between two or more sequences. Identity is measured by dividing the number of identical residues by the total number of residues and multiplying the product by 100 to achieve a percentage. Thus, two copies of exactly the same sequence have 100% identity, whereas two sequences that have amino acid deletions, additions, or substitutions relative to one another have a lower degree of identity. Those skilled in the art will recognize that several computer programs, such as those that employ algorithms such as BLAST (Basic Local Alignment Search Tool, Altschul et al. (1993) J. Mol. Biol. 215:403-410) are available for determining sequence identity.

The term “antibody” includes monoclonal antibodies (including full length monoclonal antibodies), polyclonal antibodies, multispecific antibodies (e.g. bispecific antibodies), and antibody fragments so long as they exhibit the desired biological activity or specificity.

As used herein, the term “treating” includes prophylaxis of the specific disorder or condition, or alleviation of the symptoms associated with a specific disorder or condition and/or preventing or eliminating said symptoms.

### Embodiments

Proliferating cell nuclear antigen (PCNA) protein activity has been discovered by applications to be altered in cancer cells. PCNA is a 29 kD protein (SEQ ID NO: 37) with an electrophoretic mobility equivalent to that of a 36 kDa protein. PCNA is an accessory factor required by DNA polymerase δ to mediate highly efficient DNA replication activity. The DNA synthesome purified from a malignant cell contains at least two forms of PCNA. The two species of PCNA differ significantly in their overall charge. Thus, an acidic, malignant or cancer specific, form of PCNA, caPCNA, and a basic, nonmalignant or normal, form of PCNA, nmPCNA, can be distinguished on a two-dimensional polyacrylamide gel.

Purified and recombinantly produced PCNA-dependent methyltransferase methyl esterifies one or more glutamic acid or aspartic acid residues on PCNA. Applicants have successfully isolated the methyl transferase polypeptide (SEQ ID NO: 33) and nucleic acid sequence (SEQ ID NO: 36) encoding the same. In accordance with one embodiment, a DNA sequence encoding the methyl transferase polypeptide (SEQ ID NO: 33) is provided. In particular, the DNA sequence encoding the methyl transferase polypeptide is selected from any of the following polynucleotide sequences: 1) the polynucleotide sequence of SEQ ID NO: 36; 2) a polynucleotide sequence encoding the amino acid sequence of SEQ ID NO: 33; 3) a polynucleotide sequence that hybridizes with the polynucleotide sequence of SEQ ID NO: 36 under a highly stringent condition, which also encodes a polypeptide with an activity of methyl esterifying one or more glutamic acid residues on PCNA; 4) a polynucleotide sequence encoding a polypeptide having a sequence identity over the entire length of the polypeptide of 90% and more, including for example, 95%, 96%, 97%, 98% or 99% with the polynucleotide sequence of SEQ ID NO: 33. As used herein “hybridized under a highly stringent condition” means the nucleic acids can be hybridized under “stringent” condition as defined in Maniatis T. et al. (ed.), Molecular Cloning: A Laboratory Manual 2.sup.nd ed., Cold Spring Harbor Laboratory (1989) or a similar condition thereof.

The present disclosure also encompasses a vector comprising the polynucleotide sequence described above. Such a vector may include a conventional bacterial plasmid, cosmid, phagemid, yeast plasmid, plant virus, animal virus, and any other viral vectors commonly used in the art. The vectors suitable for the invention include but are not limited to vectors used for expressing in bacteria (including all types of prokaryotic expression vectors), vectors used for expressing in yeast (such as the vectors of Pichia pastoris and Hansenula polymorpha, etc.), baculovirus vectors used for expressing in insect cells, vectors used for expressing in mammals (such as adenovirus vector, vaccinia virus vector, retrovirus vector, lentivirus vector, etc.), plant virus vectors used for expressing in plants and organ-specific expression vectors used in mammals, such as mammary expression vectors, etc. Any plasmid or vector may be used as long as they can be stably replicated and passaged in host cells. In one embodiment the expression vectors include selective marker gene, such as anti-ampicillin gene, anti-acheomycin gene, anti-kanamycin gene, anti-streptomycin gene, anti-chloramphenicol gene, etc for bacterial based vectors; anti-neomycin gene, anti-Zeocin gene for microzyme; defection selective markers, such as His, Leu, Trp, etc for microzyme; anti-neomycin gene, anti-Zeocin gene, dihydrofolacin reductase gene and fluorescin marker gene, etc for eukaryotic vectors.

Those skilled in the art are able to create the expression vectors comprising the specific elements such as DNA sequences, suitable transcription and translation sequences, promoters and selective marker genes, etc described in the invention using a series of techniques including the DNA recombination technique known in the art. The above vectors can be used to transform and transfect appropriate host cells or organisms, thereby obtaining the desired recombinant methytransferase of interest. Host cell comprising the novel nucleic acid constructs are also included in the scope of the present disclosure. Such a cell may be a prokaryotic cell or a eukaryotic cell, such as a bacterial cell, a yeast cell, a plant cell, an insect cell, a mammal cell, etc. After transformed or transfected with the inventive DNA sequence encoding the methyltransferase, the host cells may be used for producing the desired polypeptide and protein, or for administering directly.

One embodiment of the present disclosure is directed to a proliferating cell nuclear antigen (PCNA)-dependent glutamate carboxyl O-methyltransferase. More particularly, in one embodiment the methyltransferease is a human methyltransferase that methylesterifies one or more acidic amino acid residues of proliferating cell nuclear antigen (PCNA). Sequence alignments with other protein carboxyl O-methyltransferases demonstrated the existence of consensus and conserved regions within this protein, and secondary structural predictions confirm its potential to form a SAM-dependent methyltransferase fold in its C-terminus.

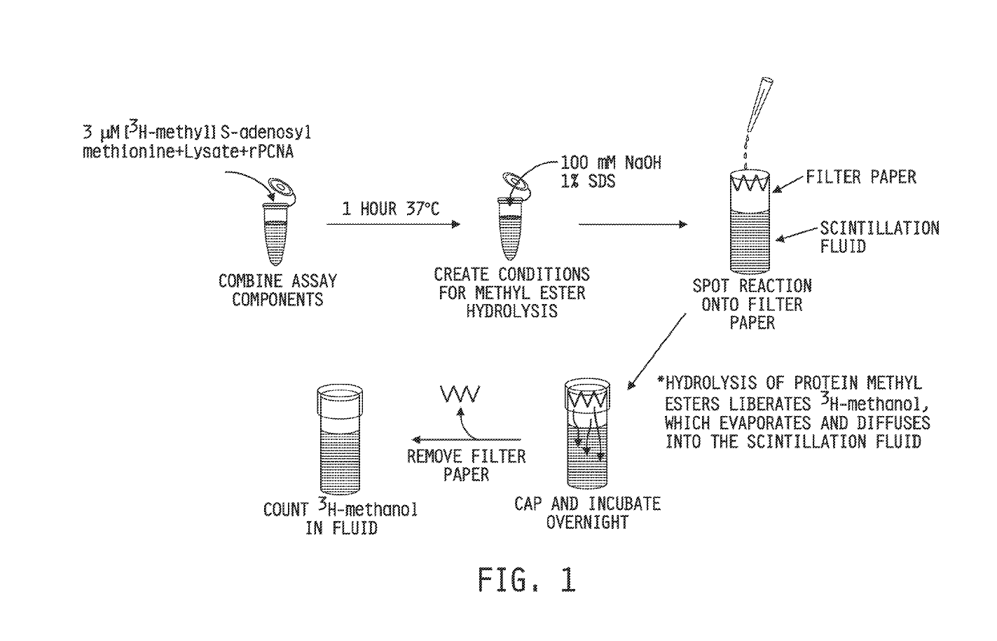

In accordance with one embodiment a purified methyltransferase is provided comprising the amino acid sequence of SEQ ID NO: 33 as well as derivatives of that sequence that retain PCNA-dependent glutamate carboxyl O-methyltransferase activity as detectable using the assay disclosed in FIG. 1. In one embodiment the PCNA-dependent glutamate carboxyl O-methyltransferase has a molecular weight of approximately 50 kDa and methylesterifies one or more acidic amino acid residues of PCNA. In one typical embodiment the methyltransferase has a secondary structure comprising 9 α-helices in its N-terminus and seven a-helices and nine β-sheets in its C-terminus. In one embodiment the methyltransferase methylesterifies one or more glutamic acid or aspartic acid residues that correspond to the amino acid positions 3, 85, 93, 94, 104, 109, 115, 120, 132, 143, 174, 189, 201, 238, 256, and 258 of the peptide of SEQ ID NO: 37.

In one embodiment a purified methyltransferase is provided comprising the sequence of SEQ ID NO: 33 or an amino acid sequence that is greater than 75%, 80%, 85%, 90%, 95% of 99% identical to the corresponding sequence of SEQ ID NO: 33 and having activity for methylesterifying one or more acidic amino acid residues of PCNA as detected using the assay disclosed in FIG. 1. In one embodiment the methyltransferease comprises an amino acid sequence that is at least about 40%, 45%, 50%, 55%, 60%, 65%, 70%, 75%, 80%, 85%, 90%, or 95% identical to the corresponding sequence of SEQ ID NO: 33. In one embodiment the methyltransferease comprises an amino acid sequence that is at least about 40%, 45%, 50%, 55%, 60%, 65%, 70%, 75%, 80%, 85%, 90%, or 95% identical to the corresponding sequence of SEQ ID NO: 33, but is identical in sequence to amino acids 245-257, 271-279, 336-347 and 356-364 of SEQ ID NO: 33. In one embodiment a methyltransferase is provided that has greater than 95% sequence identity over the entire length of the polypeptide of SEQ ID NO: 33. In one embodiment a methyltransferase is provided that is identical in sequence at amino acid positions corresponding to amino acids 245-257, 271-279, 336-347 and 356-364 of SEQ ID NO: 33, and has greater than 95% sequence identity over the entire length of the polypeptide relative to the sequence of SEQ ID NO: 33.

Modifications and substitutions described herein are, in certain aspects made at specific positions within the methyltransferase wherein the numbering of the position corresponds to the numbering of the polypeptide of SEQ ID NO: 33. In one embodiment those modifications are at non-conserved locations and in a further embodiment the modifications comprise constitutive amino acid substitutions. In accordance with one embodiment the methyltransferease comprises an amino acid derivative of SEQ ID NO: 33 wherein the peptide is modified in a nonconservative region of the peptide. For example, in one embodiment the modifications do not impact amino acids 245-257 of the methyltransferase of SEQ ID NO: 1, or other conserved regions. In accordance with one embodiment the methyltransferease comprises an amino acid sequence of SEQ ID NO: 33 wherein 1-20, 1-15, 1-10, 1-5 or 1-3 amino acids have been modified relative to the amino acid sequence of SEQ ID NO: 33. In one embodiment the modified amino acids are at a position other than amino acid positions 245-257, 271-279, 336-347 and 356-364 relative to SEQ ID NO: 33. In one embodiment, the methyltransferase is a derivative of the polypeptide of SEQ ID NO: 33, comprising a total of 1, up to 2, up to 3, up to 4, up to 5, up to 6, up to 7, up to 8, up to 9, or up to 10 amino acid modifications relative to the native methytransferase sequence, e.g. conservative or non-conservative substitutions.

In accordance with one embodiment a PCNA-dependent glutamate carboxyl O-methyltransferase is provided having a molecular weight of about of approximately 50 kDa that is at a purity level of at least 90%, 91%, 92%, 93%, 94%, 95%, 96%, 97%, 98% or 99%. In a further embodiment the methyltransferase is recombinantly expressed and is isolated away from other cellular proteins and components to a purity level of at least 90%, 91%, 92%, 93%, 94%, 95%, 96%, 97%, 98% or 99%. In one embodiment the isolated polypeptide has a secondary structure comprising 9 α-helices in its N-terminus and seven a-helices and nine n-sheets in its C-terminus. In one embodiment the methyltransferase methylesterifies one or more glutamic acid or aspartic acid residues that correspond to the amino acid positions 3, 85, 93, 94, 104, 109, 115, 120, 132, 143, 174, 189, 201, 238, 256, and 258 of SEQ ID NO: 37.

In accordance with one embodiment the isolated/purified methyltransferase of the present disclosure, when used in the assay described in Example 1 and in FIG. 1, will methylesterify one or more glutamic acid residues of the PCNA polypeptide of SEQ ID NO: 37. In accordance with one embodiment the methyltransferase, when exposed to the peptide of SEQ ID NO: 37, or a fragment thereof (e.g. SEQ ID NOs. 16-30 and 32) in the presence of S-adenosyl-L-methionine produces a peptide comprising one or more of the following sequences:

(SEQ ID NO: 1)
MFEₘAR;
(SEQ ID NO: 2)
IEₘDEEGS;
(SEQ ID NO: 3)
IEDEEₘGS;
(SEQ ID NO: 4)
VSDYEₘMK;
(SEQ ID NO: 5)
MPSGEₘFAR;
(SEQ ID NO: 6)
LSQTSNVDₘK;
(SEQ ID NO: 7)
CAGNEₘDIITLR;
(SEQ ID NO: 8)
FSASGEₘLGNGNIK;
(SEQ ID NO: 9)
AEDNADTLALVFEAPNQEₘK;
(SEQ ID NO: 10)
AEₘDNADTLALVFEAPNQEK;
(SEQ ID NO: 11)
AEDₘNADTLALVFEAPNQEK;
(SEQ ID NO: 12)
AEDNADTLALVFEₘAPNQEK;
(SEQ ID NO: 13)
LMDₘLDVDQLGIPEQEYSCVVK;
(SEQ ID NO: 14)
ATPLSSTVTLSMSADVPLVVEₘYK;
(SEQ ID NO: 15)
LSQTSNVDKEEEAVTIEMNEₘPVQLTFALR;
and
(SEQ ID NO: 31)
LMDLDVEQLGIPEQEₘYSCVVK

The unique methyltransferase of SEQ ID NO: 33 is involved in DNA replication and repair. As disclosed herein the activity of the enzyme is also correlated with cancer, and decreased activity of the enzyme can be used as a diagnostic marker for cancer, including breast cancer.

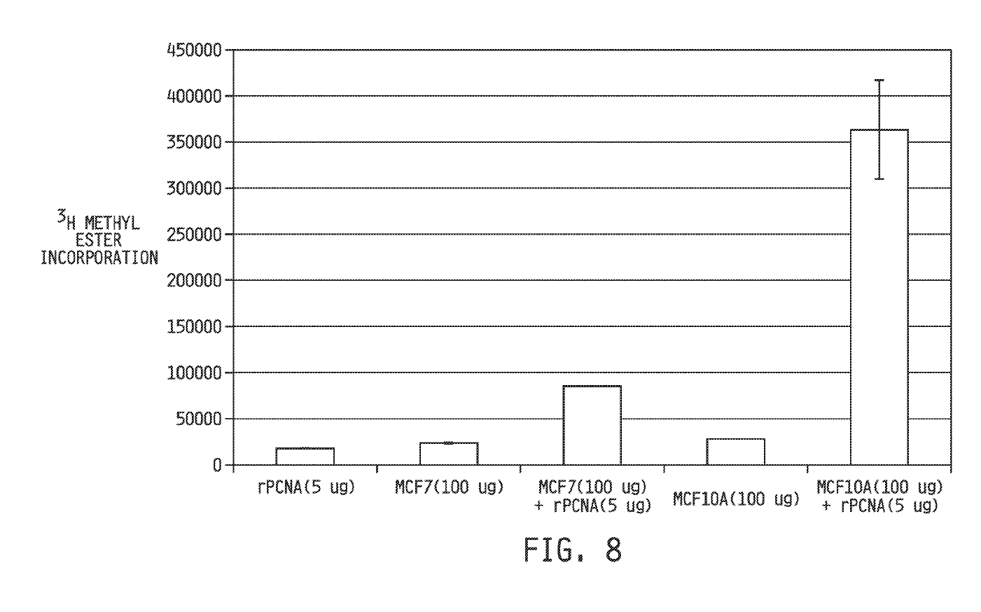

As applicants have demonstrated in the data presented in Example 2 and in FIG. 8, that non-malignant cells exhibit higher levels of methyltransferase activity. The acidic form of caPCNA is expressed in malignant cell lines, such as HeLa (human cervical carcinoma), Hs578T (breast carcinoma), HL-60 (human promyelogenous leukemia), FM3A (mouse mammary carcinoma), PC 10 (prostate carcinoma), LNCaP (prostate carcinoma), LN99 (prostate carcinoma) MD-MB468 (human breast carcinoma), MCF-7 (breast carcinoma), KGE 90 (esophageal-colon carcinoma), KYE 350 (esophageal-colon carcinoma), SW 48 (esophageal-colon carcinoma) and T98 (malignant glioma). The acidic caPCNA is also expressed in malignant cells obtained from human breast tumors, prostate tumors, brain tumors, human gastrointestinal or esophageal-colon tumors, murine breast tumors and in human chronic myelogenous leukemia. The acidic caPCNA is not detected in nonmalignant cell lines, such as the breast cell lines Hs578Bst and MCF-10A, or in samples of nonmalignant serum or tissue, such as breast.

An LC-MS/MS peptide characterization approach was used to sequence a caPCNA isoform found in malignant cells. A novel type of post-translational modification present on numerous residues of caPCNA was identified. This modification, methyl esterification, was present on 16 different aspartic acid and glutamic acid residues in caPCNA. These methyl esters were initially identified as 14 Da shifts in peptide mass and were localized to either glutamic or aspartic acid residues by tandem mass spectrometry. Relative quantitation of the methyl esterified peptides indicated that caPCNA proteins in malignant cells include several molecules containing one or more methyl esters that occur at multiple residues throughout the protein. Methyl esterification of specific residues is likely to result in discrete conformational changes in the protein, and these changes may promote and/or disrupt protein/protein interactions.

The effects of methyl esterification on mammalian protein functions are poorly understood. Much of the past research into methyl esterification of mammalian proteins has focused on protein aging and the repair of isoaspartyl residues by the enzyme protein isoaspartate methyl transferase (PIMT). However, most methyl esters present on caPCNA are found on glutamic acid residues and not aspartic acid residues suggesting that the modification occurs via an alternate pathway.

It is anticipated that methyl esterification of PCNA alters its conformation and, in effect, hide and/or expose specific protein binding sites and determines its function. LC-MS/MS sequence analysis of recombinant PCNA was also performed and evidence for methyl esterification was found. The methyl esterification found on PCNA may therefore stabilize specific conformational states of an otherwise disordered protein. Additionally, calculation of the electrostatic potential of PCNA shows that the outer surface of the PCNA trimer has a highly negative potential and an abundance of glutamic and aspartic acid residues. Methylation of these residues could therefore alter this potential and, in effect, change the surface topology of the protein.

Detection of the altered form of PCNA, or diminished methyltmasferase activity in a patient's biological sample can be diagnostic for cancer. The biological sample can be a body fluid sample, which may include blood, plasma, lymph, serum, pleural fluid, spinal fluid, saliva, sputum, urine, semen, tears, synovial fluid or any bodily fluid that can be tested for the presence of the caPCNA isoform or methyltransferase activity. Alternatively, the biological sample can be a tissue sample, wherein the cells of the tissue sample may be suspected of being malignant. For example, tissue or cell samples can by lysed and their lysates can be used to measure methyltransferase activity using the assay of Example 1, FIG. 1, for example. However any assay capable to detecting and quantitaing the amount of methyesterification of the target PCNA peptide (or fragment thereof) can be used in accordance with the diagnostic assay as disclosed herein. Tissue extracts or concentrates of cells or cell extracts are also suitable.

In accordance with one embodiment a method for diagnosing the presence of cancer or a pre-cancerous condition is provided. The method comprises determining the level of activity of the proliferating cell nuclear antigen (PCNA)-dependent glutamate carboxyl O-methyltransferase and determining if that activity falls below a threshold level to indicate a risk of cancer. In accordance with one embodiment the threshold level can be determined based on population data wherein the average amount of (PCNA)-dependent glutamate carboxyl O-methyltransferase activity is established. The population data can be tailored to the individual by accounting for age, ethnicity, sex and other parameters. Alternatively, two biological samples can be obtained from the patient to be screen for cancer. The first sample may be taken from tissue suspected to be precancerous whereas the second sample can be taken from healthy tissue. In one embodiment the two biological samples are recovered from the same tissue type. The levels of (PCNA)-dependent glutamate carboxyl O-methyltransferase activity is determined for the two biological samples wherein a substantial decrease in methylase activity in the test sample relative to the sample recovered from healthy tissue is indicative of cancer or a precancerous condition.

In one embodiment a method for diagnosing cancer or a method of determining the effectiveness of anti-cancer treatment is provided. The method comprises obtaining a series of biological test samples from a patient during the course of the anti-cancer treatment. The samples are then analyzed for determining the relative levels of proliferating cell nuclear antigen (PCNA) targeted methyltransferase activity. Effectiveness of the treatment is indicated by increased levels of methyltransferase activity.

In another embodiment, a method for diagnosing malignancy is provided. The method comprises the step of detecting PCNA-dependent methyltransferase (MTT) activity in a biological sample obtained from a person or particularly a patient suspected of having a malignant condition, wherein the step of detecting levels of PCNA-dependent methyltransferase (MTT) activity also optionally involves detecting posttranslational modification of PCNA. In one embodiment an antibody specific for caPCNA is used to detect posttranslational modification of PCNA.

In another embodiment, a method to aid in diagnosing malignancy is provided. The method comprises the step of detecting MTT levels or expression of caPCNA in a tissue sample compared to normal cells, wherein cells of the tissue sample are suspected of being malignant. Optionally, the detecting caPCNA step further involves detecting methyl esters on caPCNA. It is to be understood that the malignant cells include, but are not limited to, malignant cells in tissues such as breast, prostate, blood, brain, pancreas, smooth or striated muscle, liver, spleen, thymus, lung, ovary, skin, heart, connective tissue, kidney, bladder, intestine, stomach, adrenal gland, lymph node, or cervix, or in cell lines, for example, Hs578T, MCF-7, MDA-MB468, HeLa, HL60, FM3A, BT-474, MDA-MB-453, T98, LNCaP, LN 99, PC 10, SK-OV-3, MKN-7, KGE 90, KYE 350, or SW 48.

In another embodiment, a method to aid prognosis of the development of malignancy is provided. The method involves detecting PCNA-dependent MTT activity in a tissue sample, wherein cells of the tissue sample may be suspected of being malignant, and correlating the levels of PCNA-dependent MTT activity with the progression of a particular malignant disease. Furthermore, the detection PCNA-dependent MTT activity and analysis of posttranslational modifications on caPCNA can be used to prognose the potential survival outcome for a patient who has developed a malignancy. In a further embodiment the PCNA-dependent MTT activity can be monitored over the course of an anti-cancer therapy as a means of measuring the efficacy and dosage of the administered therapeutic.

It is to be understood that the diseases which can be diagnosed or prognosed using the antibodies include, but are not limited to, malignancies such as various forms of glioblastoma, glioma, astrocytoma, meningioma, neuroblastoma, retinoblastoma, melanoma, colon carcinoma, lung carcinoma, adenocarcinoma, cervical carcinoma, ovarian carcinoma, bladder carcinoma, lymphoblastoma, leukemia, osteosarcoma, breast carcinoma, hepatoma, nephroma, adrenal carcinoma, or prostate carcinoma, esophageal carcinoma. If a malignant cell expresses caPCNA isoform, the techniques disclosed herein are capable of detecting the PCNA-dependent MTT activity.

Detection techniques involving the detection of PCNA-dependent MTT activity disclosed herein, could also detect malignancy in some of the invasive and non-invasive tumor types in breast tissue that includes ductal cysts, apocrine metaplasia, sclerosing adenosis, duct epithelial hyperplasia, non-atypical, intraductal papillomatosis, columnar cell changes, radial sclerosing lesion (radial scar), nipple adenoma, intraductal papilloma, fibroadenoma, lactating papilloma, atypical duct epithelial hyperplasia, atypical lobular hyperplasia, ductal carcinoma in situ—sub classified as nuclear grades 1, 2, and 3, lobular carcinoma-in-situ, pleomorphic lobular carcinoma-in-situ, intra-mammary lipoma, mammary hamartoma, granular cell tumor, intramammary fat necrosis, pseudoangiomatous stromal hyperplasia (PASH), malignant melanoma involving the breast, malignant lymphoma involving the breast, phyllodes tumor—benign, borderline, and malignant subclasses, and sarcoma of the breast.

In another embodiment, methods disclosed herein are used to determine the malignancy stage in tumors, by comparing levels of PCNA-dependent MTT activity in a tumor over time, to follow the progression of a malignant disease, or a patient's response to treatment. The methods can also be used to detect malignant cells which have broken free from a tumor and are present in a patient's bloodstream, by methods to assay a blood sample for the presence of the caPCNA isoform. The biological sample can be obtained from human patients or veterinary patients.

In accordance with one embodiment a patient's biological sample is analyzed for the relative amount of PCNA-dependent MTT activity present in the cells of the tissue being analyzed. This measurement is then compared to a standard, wherein PCNA-dependent MTT activity below a certain threshold level is indicative of either the presence of cancer or an elevated risk of cancer, or the existence of precancerous cells. The threshold value can be generated based on population data relating to measured PCNA-dependent MTT activity in normal healthy tissues. In one embodiment average PCNA-dependent MTT activity will be established for a variety of tissue and cell types as well as controlling for other factors such as age, ethnicity, sex and the like. In an alternative embodiment the standard may comprise a measurement of PCNA-dependent MTT activity in a second sample taken from the same patient as the original test sample, but from healthy tissues.

The step of detecting the PCNA-dependent MTT activity in the biological samples can be conducted using any technique known to those skilled in the art. For example, mass spectrometric analyses is a suitable technique. Mass spectrometric analysis can also be coupled with other techniques. Antibodies that specifically recognize posttranslationally modified nmPCNA or caPCNA can be made. Accordingly, immunoassays can be used to detect changes in posttranslationally modified nmPCNA or caPCNA as a means of measuring PCNA-dependent MTT activity. As a further example, the assay disclosed in FIG. 1 can also be used to quantitate PCNA-dependent MTT activity.

In one embodiment a kit is provided for conducting methylesterification assays to measure the level of methyltransferase activity in a sample. In one embodiment the kit comprises a PCNA-dependent MTT and a substrate for the methyesterification. In one embodiment the PCNA-dependent MTT is the novel proliferating cell nuclear antigen (PCNA)-dependent glutamate carboxyl O-methyltransferase disclosed herein. In one embodiment the PCNA-dependent MTT is a polypeptide comprising the amino acid sequence of SEQ ID NO: 33, or a modified derivative thereof. In one embodiment the methyltransferase substrate comprises an amino acid sequence of SEQ ID NO: 37 (i.e., proliferating cell nuclear antigen (PCNA)), or a peptide comprising an amino acid sequence selected from the group consisting of

(SEQ ID NO: 16)
MFEAR;
(SEQ ID NO: 17)
IEDEEGS;
(SEQ ID NO: 18)
IEDEEGS;
(SEQ ID NO: 19)
VSDYEMK;
(SEQ ID NO: 20)
MPSGEFAR;
(SEQ ID NO: 21)
LSQTSNVDK;
(SEQ ID NO: 22)
CAGNEDIITLR;
(SEQ ID NO: 23)
FSASGELGNGNIK;
(SEQ ID NO: 24)
AEDNADTLALVFEAPNQEK;
(SEQ ID NO: 25)
AEDNADTLALVFEAPNQEK;
(SEQ ID NO: 26)
AEDNADTLALVFEAPNQEK;
(SEQ ID NO: 27)
AEDNADTLALVFEAPNQEK;
(SEQ ID NO: 28)
LMDLDVEQLGIPEQEYSCVVK;
(SEQ ID NO: 29)
ATPLSSTVTLSMSADVPLVVEYK;
(SEQ ID NO: 30)
LSQTSNVDKEEEAVTIEMNEPVQLTFALR;
and
(SEQ ID NO: 32)
LMDLDVEQLGIPEQEYSCVVK.

In one embodiment the kit further comprises comprising S-adenosyl-L-methionine. The kit may further include a variety of containers, e.g., vials, tubes, bottles, and the like. Preferably, the kits will also include instructions for use.

While the methods of determining PCNA-dependent MTT activity and detecting posttranslational modification and uses thereof relating to the caPCNA isoform have been described in detail in the detailed description and in the Examples below, and with reference to specific embodiments thereof, it will be apparent to one with ordinary skill in the art that various changes and modifications can be made therein without departing from the spirit and scope thereof All references cited herein are incorporated by reference in their entirety.

### EXAMPLES

The following examples are provided for the purpose of exemplification only and are not intended to limit the disclosure which has been described in broad terms above.

### Example 1

### Dentification of a PCNA-Dependent Carboxyl Methyltransferase—ARM1

In eukaryotic cells, there are three known protein carboxyl methyltransferases classified according to their substrates. The most widely known eukaryotic enzyme is the protein repair factor protein isoaspartate methyltransferase (PIMT). PIMT is responsible for methyl esterifying and repairing iso-aspartate residues in aging proteins. Protein leucine carboxyl methyltransferase (LCMT) represents a second class of eukaryotic carboxyl methyltransferases and its substrate is the C-terminal leucine of the tumor suppressor protein phosphatase 2A (PP2A). The last eukaryotic enzyme is isoprenylcysteine carboxyl methyltransferase (ICMT), which is responsible for methyl esterifying C-terminal cysteines of membrane associated proteins during post-prenylation processing. Although the exact function of ICMT is not fully understood, it has shown promise as a therapeutic target for cancer therapy suggesting it may have an important role in tumor cell growth.

A fourth class of protein carboxyl methyltransferases is limited to prokaryotic cells and targets glutamyl residues in proteins. In motile bacteria, chemotaxis receptors are targets of the protein glutamyl methyltransferase, CheR. CheR methyl esterifies four glutamic acid residues in membrane receptors following ligand binding promoting its interaction with an intracellular kinase thus activating a signal transduction cascade that alters bacterial swimming.

Methods

Vapor diffusion assay. The assay was performed basically as previously described (Murray, E. D., Jr. & Clarke, S. J Biol Chem 259, 10722-10732 (1984). MCF7 breast cancer cell extracts were assayed 1-2 h in the presence of [³H-methyl]-SAM (NEN). Following incubation, extracts were equilibrated with 100 mM NaOH with 2% SDS and spotted onto filter paper folded into an accordion pleat and placed into the neck of a vial above scintillation fluid. ³H-methanol present in the scintillation fluid was measured the following day (See FIG. 1).

Protein expression and purification. HiTrap phenyl Sepharose HP and Superdex S200 columns were purchased from GE Biosciences. Chromatography was performed using a Biologic DuoFlow (BioRad). Recombinant PCNA was expressed as a calmodulin binding protein (CBP) fusion using the pDual expression system and purified using calmodulin (Stratagene) or as a 6× His-tagged fusion (Origene) and purified with MagneHis-Ni particles. The CBP tag lacks both aspartic and glutamic acids. Full-length p21 and the p21 PIP domain GST fusions were expressed in a pGEX-2TK vector and purified on glutathione Sepharose (GE Biosciences). Full-length p21 was isolated from inclusion bodies as described. (Podust et al., Biochemistry 34, 8869-8875 (1995). A eukaryotic expression vector expressing a Flag-Arm1 fusion was transfected into and transiently expressed in SK-Br-3 cells with Fugene 6 (Roche). Anti-Flag immunoprecipitations were performed with Anti-Flag M2 Affintiy Gel (Sigma) and detected by Western analysis using anti-DDK antibodies (Origene). PIP-affinity beads were generated by covalently coupling synthetic p21 PIP peptide (Anaspec) to CH Sepharose (GE Biosciences).

2D-PAGE and mass spectrometry. Isoelectric focusing was performed using IPG strips and an IEF cell (BioRad) as previously described (Hoelz, D. J. et al. Proteomics 6, 4808-4816, 2006). Protein identifications were achieved with an LCQAdvantage mass spectrometer equipped with a nanospray source and LC-MS/MS data analyzed against the SwissProt mammalian protein database with Mascot. Anti-PCNA (PC10) antibodies were obtained from Santa Cruz Biotech and anti-WAF1 (DF10) were from EMD Biosciences.

Results

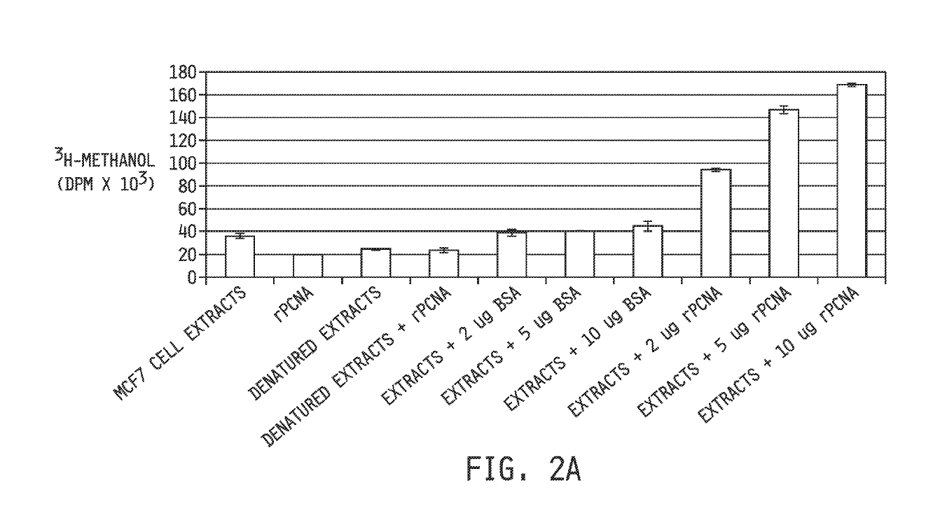

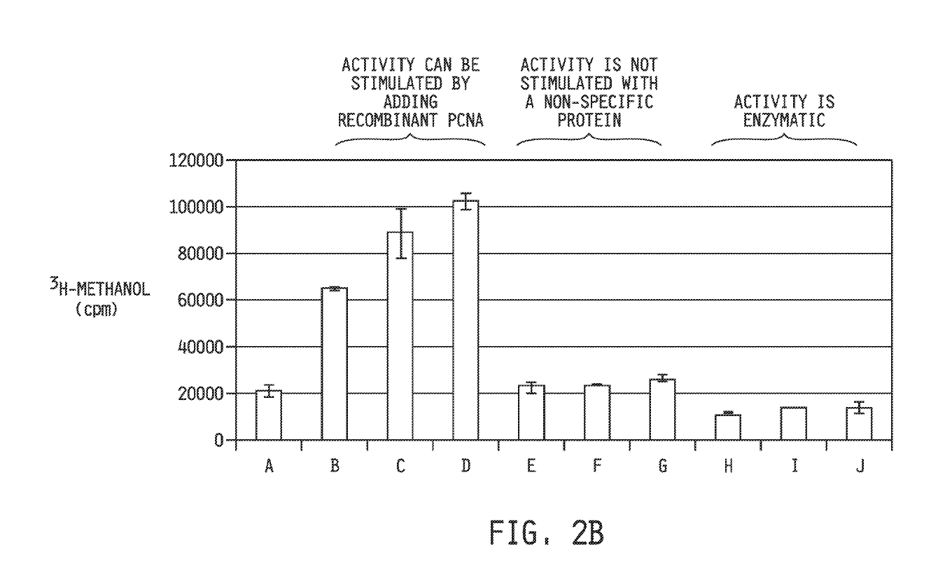

To identify PCNA-dependent carboxyl methyltransferase activity in human cells, breast cancer cell extracts were assayed in the presence of radiolabeled-S-adenosyl-L-methionine (SAM). Methyl esters formed by SAM-dependent methyltransferase activity were hydrolyzed by the addition of base and the liberated radiolabeled methanol was measured by passive diffusion into scintillation fluid (See FIG. 1). Using this approach applicants detected enzymatic methyl esterification of substrates endogenous to the breast cancer cell extracts (FIG. 2A and FIG. 2B). Addition of exogenous purified recombinant PCNA to the assay significantly increased the amount of methyl esters detected in a in a dose-dependent fashion, and 10 μg increased the amount of methyl esters detected by nearly five-fold over background. Addition of BSA, a non-specific protein, failed to elevate methyl ester levels. These results both indicated that PCNA was a target of a carboxyl methyltransferase in human cells and suggested that it was not likely a generalized phenomenon such as protein repair. These data also confirmed initial observations upon detailed protein sequencing that PCNA was methyl esterified on 15-16 specific glutamic and aspartic acid residues (Hoelz, D. J. et al. Proteomics 6, 4808-4816, 2006).

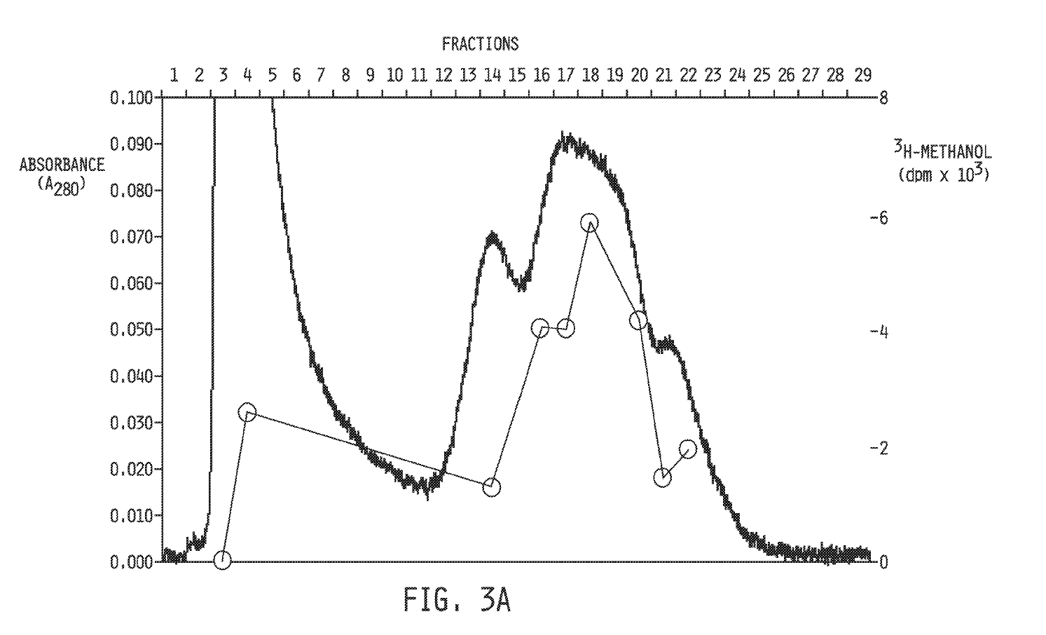

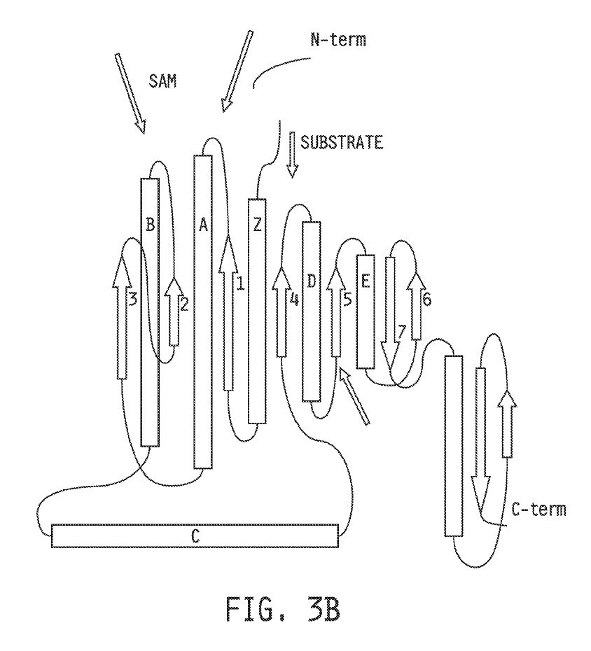

Using this assay applicants then enriched for PCNA-dependent activity in human breast cancer cell extracts (FIG. 3). Cell extracts were equilibrated with 30% NH₄SO₄ and the soluble fraction loaded onto a phenyl Sepharose column. PCNA-dependent carboxyl methyltransferase activities were eluted with a linear gradient of NH₄SO₄ (30-0%) and fractions assayed for PCNA-dependent methyltransferase activity. Activity was further enriched from pooled phenyl Sepharose fractions by passage over a Superdex S200 gel filtration column, and the active fractions resolved by 2D-PAGE. Proteins were excised from the 2D-PAGE gel, digested with trypsin and analyzed by LC-MS/MS. The proteins were identified using proteomics techniques and were grouped by cellular function. The majority of proteins identified using this approach were of known function and could be excluded. The bulk of proteins were metabolic or cytoskeletal, but chaperones, translational initiation factors, cell signaling, and splicing factors were also observed. The field of methyltransferase candidates was narrowed down to two protein products of hypothetical orfs. Of these two unknown orfs, one encoded a protein small in size (10 kDa) and limited in structures common to the SAM-dependent methyltransferases. The other unknown protein, however, was the product of the 211th hypothetical orf on chromosome 6 (C6orf211). The C6orf211 protein possessed a mass of 50 kDa (supplementary FIG. 1), and was an attractive candidate a for a potential PCNA-dependent carboxyl methyltransferase.

To further examine the product of this hypothetical orf the protein sequence was aligned with both the E. coli glutamyl methyltransferase CheR and the human protein repair enzyme protein iso-aspartate methyltransferase (PIMT) (see FIG. 4). Although these alignments revealed limited sequence conservation among all three proteins, it is characteristic of this family of enzymes. Despite significant sequence diversity, the SAM-dependent methyltransferases all share a common tertiary structure referred to as the SAM-dependent methyltransferase (SAM-MT) fold (Martin,et al., Curr Opin Struct Biol 12, 783-793, 2002).

The SAM-MT core fold consists of seven β-sheets that alternate with five α-helices producing the binding pockets for both SAM and its substrate. Analysis of the C6orf211 gene product predicted secondary structures in the C6orf211 protein and identified 9 α-helices in its N-terminus and a collection of seven a-helices and nine β-sheets in its C-terminus. As a comparison, CheR consisted of four N-terminal α-helices (responsible for interacting with the chemotaxis receptor12) and six α-helices and ten β-sheets in its C-terminus forming the SAM-MT fold. Using the predicted secondary structures in the C-terminus of the C6orf211 gene product a hypothetical SAM-MT fold was proposed (FIG. 3B).

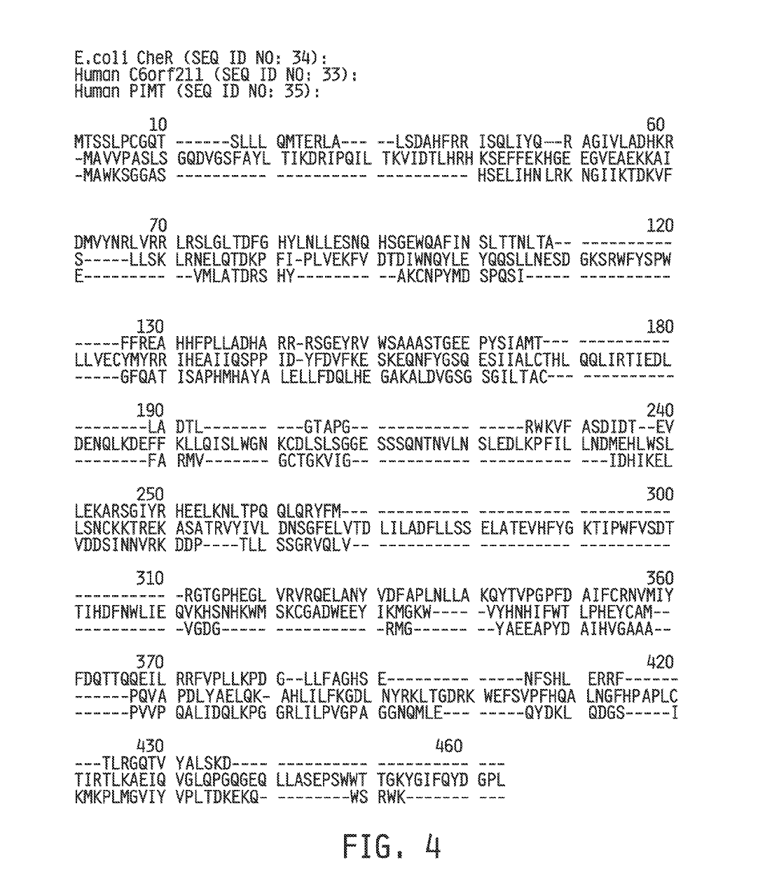

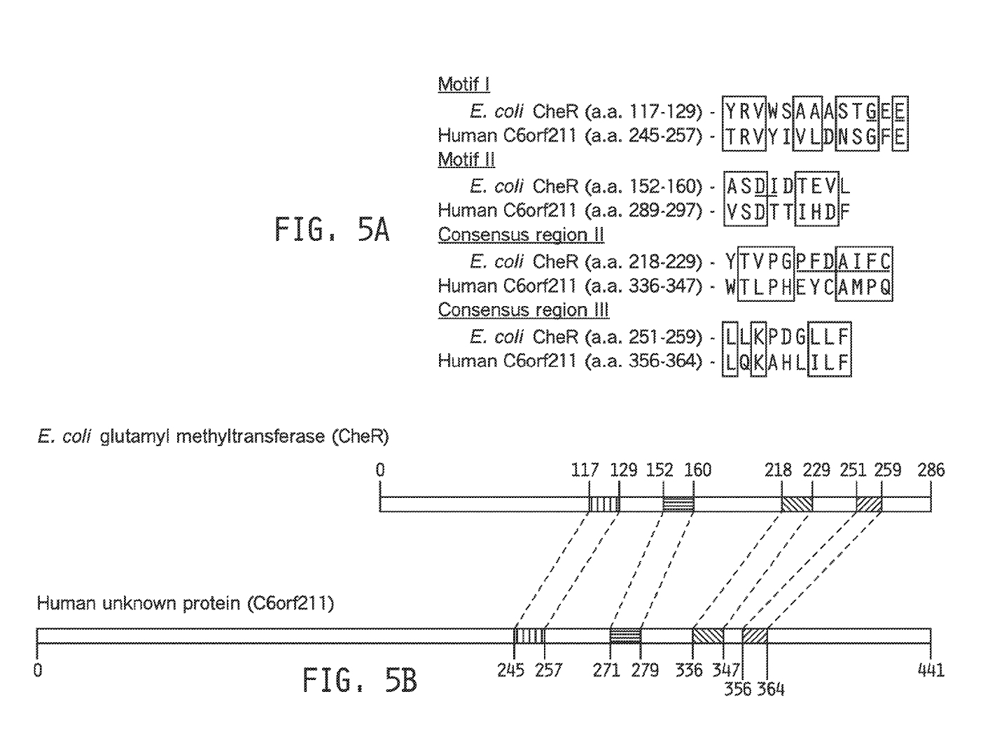

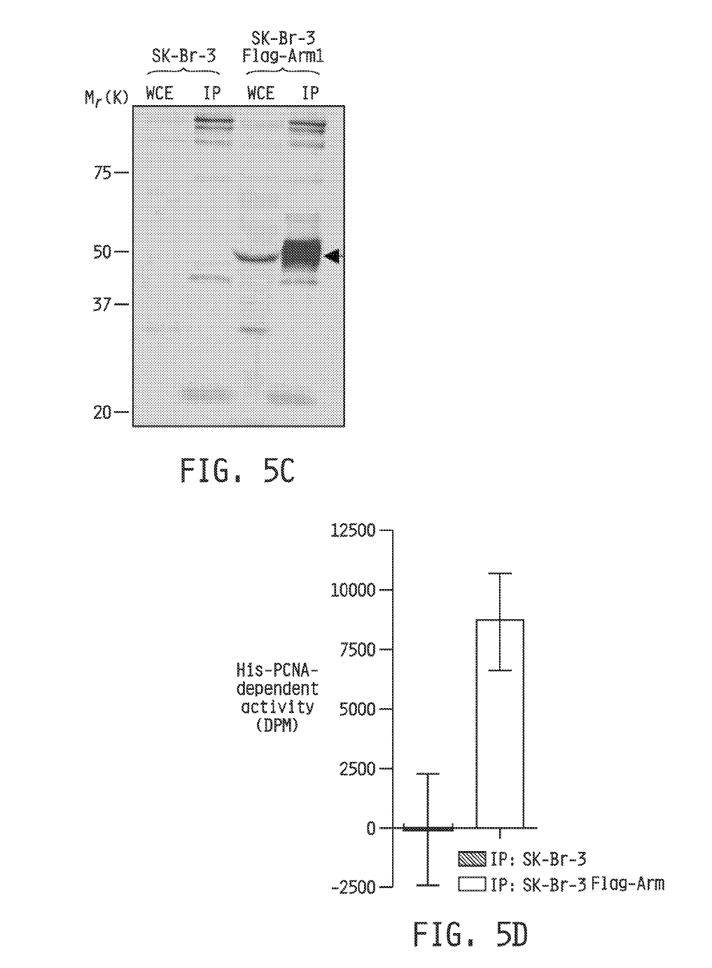

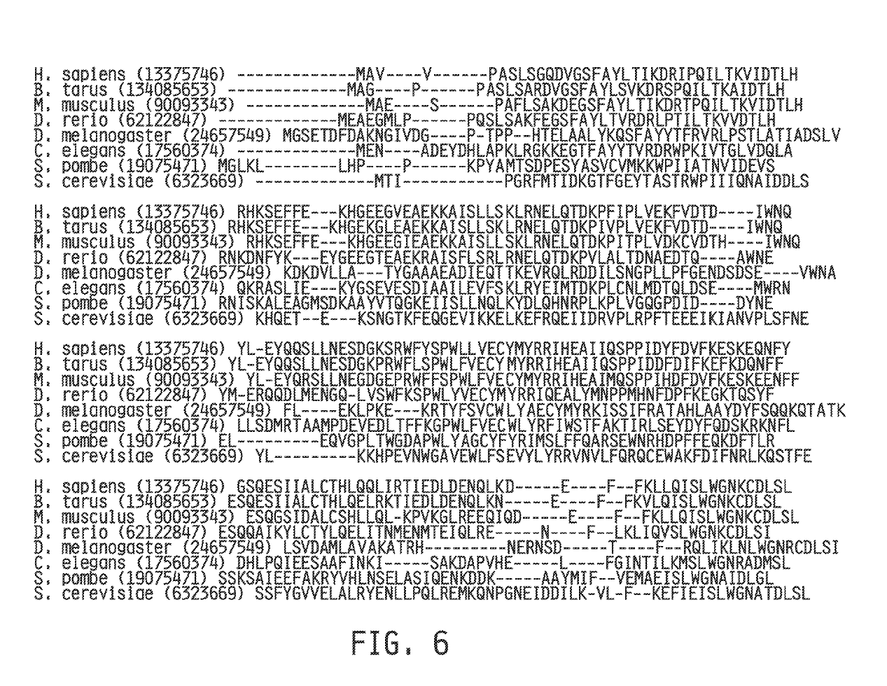

Within the SAM-MT fold two highly conserved motifs have been identified that create the SAM binding pocket. Motif I is present within the β1/αA loop of the SAM-MT fold interacts with the amino acid portion of the SAM molecule. Alignment of the CheR motif I sequence with the full-length C6orf211 gene product identified amino acids 245-257 in its C-terminus with significant homology (FIG. 5A). These aligned sequences identified a conserved glutamic acid and glycine residue, the latter of which is conserved throughout all of the SAM-MTs with a single exception, the RNA-dependent methyltransferase VP39. Evolutionary alignments of the C6orf211 gene products from eight different eukaryotic species (FIG. 6) identified high conservation in this sequence and 100% conservation of the glutamic acid and glycine residues suggesting that these residues are essential for the protein's function. Furthermore, positioning of this sequence in the hypothetical SAM-MT fold placed it precisely in the β1/αA loop. Motif II forms an acidic loop between β2 and αB that hydrogen bonds with the ribose hydroxyls of SAM. Inspection of the C6orf211 protein alignments (FIG. 6) identified a highly conserved acidic sequence (FIG. 5A) present in the β2/αB loop of the hypothetical SAM-MT structure and 32 residues C-terminal to motif I.

In addition to motifs I and II, two less conserved regions (II and III) not required for SAM binding have also been identified in CheR and PIMT11. The C6orf211 protein was also examined for these sequences (FIG. 5A). Using an alignment tool that identifies homologous sequences in distantly related proteins the CheR and C6orf211 protein sequences were aligned as shown in FIG. 5. In addition to the presence of sequences similar to motifs I and II and regions II and III, their relative positions within the C6orf211 protein were also close to those of CheR (FIG. 5B). Taken together these results strongly supported a SAM-dependent methyltransferase function for the C6orf211 gene product.

To confirm these results, applicants examined the ability of C6orf211 protein to modify PCNA in vitro. A Flag-tagged C6orf211 protein fusion (Flag-Arm1) was expressed in SK-Br-3 breast cancer cells, and using anti-Flag antibodies, the Flag-Arm1 protein was isolated (FIG. 5C). Using the vapor diffusion assay, the protein's ability to methyl esterify purified recombinant PCNA was investigated (FIG. 5D). Significant levels of PCNA-dependent carboxyl methyltransferase activity was detected in the anti-Flag Flag-Arm1 expressing extracts, and this activity was not present in the immunoprecipitates from control extracts. These results establish the C6orf211 gene product as a PCNA-dependent carboxyl methyltransferase, now designated as acidic residue methyltransferase 1 (Arm1).

The p53 and p21-Dependent Methyl Esterification of PCNA

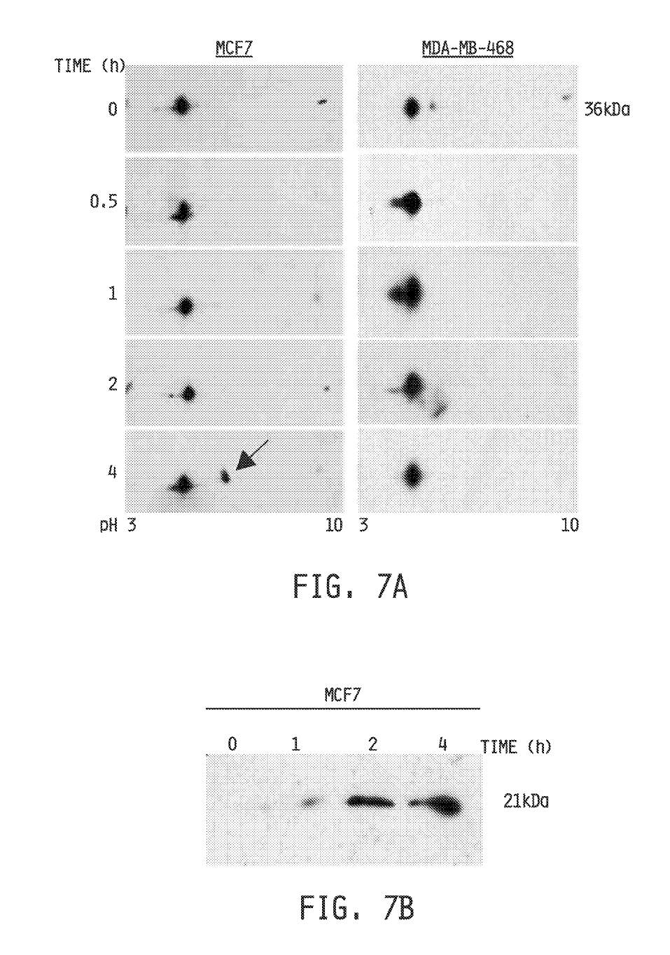

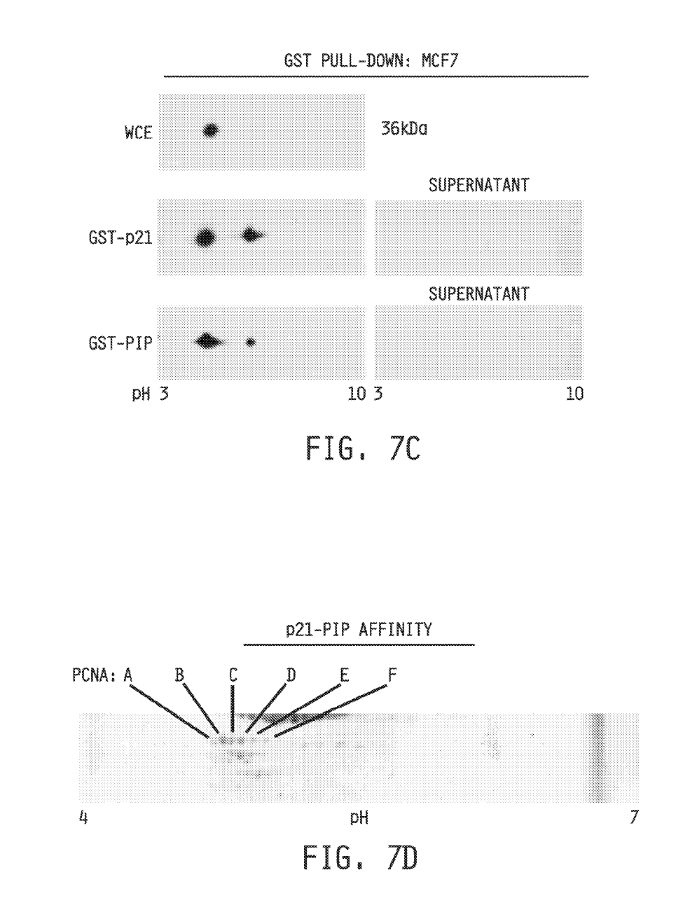

Although the description of Arm1 identifies a novel posttranslational mechanism in eukaryotic cells, its biological significance was still unclear. Given PCNA's established roles in DNA repair and DNA damage tolerance, applicants examined PCNA methyl esterification in breast cancer cells following exposure to the genotoxic agent doxorubicin (DOX) (FIG. 7A). By monitoring PCNA's isoelectric point (pI) with 2D-PAGE, an isoform was identified in MCF7 cells induced following high-dose DOX treatment. This isoform displayed a pI similar to the calculated value for PCNA devoid of 16 acidic charges (pH˜5.6), the number of methyl esterified glutamic and aspartic acidic residues initially found on PCNA2. This isoform, appeared 4 h after exposure to exposure to DOX was relatively short-lived lasting ˜15 m (data not shown). To determine whether this event was specific to MCF7 cells, we examined another breast cancer cell line MDA MB468, and were unable to identify the isoform (FIG. 7A). In contrast to MCF7 cells, however, MDA MB 468 cells harbor an inactivating mutation17 in the p53 tumor suppressor, a key mediator of the DNA damage response. It was therefore possible that the absence of this PCNA isoform was the result of absent p53 function. In response to DNA damage, p53 wild-type cells induce the cyclin-dependent kinase inhibitor p21WAF1/CIP1 while p53-mutant cells do not, and in response to DOX treatment MCF7 cells induced p21 expression over 27-fold 4 h after DOX treatment (FIG. 7B). In addition to inhibiting cell cycle progression, p21 also binds to PCNA and inhibits DNA replication in response to DNA damage. The appearance of this PCNA isoform in MCF7 and not MDA MB468 cells could therefore have been due to the presence and absence of p21. As a result applicants examined the interaction of PCNA with p21 in breast cancer cells.

Using a GST-p21 fusion applicants isolated PCNA from untreated breast cancer cell extracts and examined its posttranslational state with 2D-PAGE gels (FIG. 7C). As expected GST-p21 pulled-down all PCNA from these extracts, but unexpectedly we observed two isoforms in the pull-down fractions. In addition to the isoform present in the input extracts we consistently observed the same basic shifted isoform identified in the p53 wild-type MCF7 cells at the 4 h DOX time point in the pull-down fractions. This suggested that the interaction of p21 with PCNA led directly to the methyl esterification of PCNA. In addition to full-length p21, applicants also examined the interaction of p21's PCNA interacting peptide (PIP), with PCNA from these extracts (FIG. 7C). Consistent with full-length p21, the basic-shifted PCNA isoform was also observed in the GST-PIP pull-down fractions suggesting that this minimal sequence of p21 was sufficient to promote the basic shift to PCNA. A p21-PIP peptide affinity approach was then developed to isolate PCNA isoforms and allow examination of the resulting PCNA isoforms for the presence of methyl esters using LC-MS/MS sequencing (FIG. 7D). Using this approach peptide affinity approach to isolate PCNA applicants observed multiple PCNA isoforms migrating toward the basic side of the 2D-PAGE gels (FIG. 7D). Although the facile lability of the methyl esters made their identifications challenging, especially in lower abundance spots, LC-MS/MS sequencing did identify a trend of increasing methyl esters on PCNA with the most basic isoforms showing the highest abundance of methyl esterified residues (table I). Consistent with this trend, two methyl esterified residues on PCNA's acidic C-terminus were observed. Although applicants were unable to detect the C-terminus in two of the spots, singly methyl esterified and unmodified C-termini were not observed in any of the spots.

Discussion

The results described herein confirm the isolation of a novel eukaryotic carboxyl methyltransferase, Arm1, which modifies glutamic and aspartic acid residues in PCNA. The data also provides evidence that PCNA methyl esterification is stimulated following exposure of cells to genotoxic stress, and that this response is mediated through p53-dependent up regulation of p21 and it's binding to PCNA. The effect methyl esterification is believed to alter the conformation of PCNA and its protein-protein interactions. Like yeast, human cells also ubiquitylate PCNA on conserved residue K164 following DNA damage, which directs its interactions to the translesion DNA polymerases. Interestingly, p53 has been shown regulate PCNA ubiquitylation and translesion DNA synthesis, and this appears to occur through binding of p21-PIP domain. It is currently unclear if PCNA methyl esterification affects ubiquitylation, but examination of the positions of methyl esters on the PCNA crystal structure complexed with the p21-PIP region20 revealed a concentration of the methyl esters in the regions responsible for trimerization. This suggested that PCNA clamp assembly may altered upon methyl esterification leading to a proposed model for PCNA trimer disassembly following p21 binding.

### Example 2

### PCNA-Dependent Methyl Transferase Activity

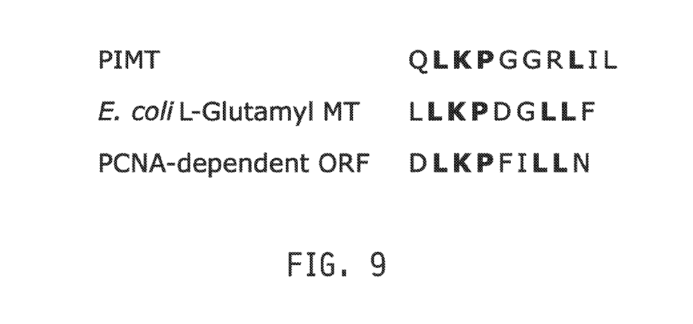

This example demonstrates that PCNA-dependent methyl transferase performs methyl esterification at one or more amino acid locations. A methyl transferase reaction was conducted as described in Example 1 using cell extracts from a breast cancer cell line (MCF7) or from a non-cancer cell line (MCF 10A) in the presence of recombinant PCNA. FIG. 8 shows that non-malignant breast cells contain higher levels of MT activity. FIG. 9 shows an amino acid sequence alignment of putative PCNA-dependent methyltransferase partial sequence ORF with known methyltransferase domains. The PCNA methyltransferase sequence aligns to the bacterial glutamate methyltransferase.

The caPCNA isoform contains a low amount of methyl esterification compared to the normal or non-malignant form of PCNA (nmPCNA or simply PCNA). The non-malignant or basic PCNA isoform likely contains a higher level of methyl esterification. This conclusion is based, in part, on the fact that methyl esterification modifies acidic residues and would shift the protein to a more basic pI (due to loss of acidic charge) and the caPCNA isoform is very close to its calculated pI of 4.5. However, acetylation, phosphorylation and ADP-ribosylation would shift a protein to a more acidic pI below 4.5 (due to addition of an acidic charge). Therefore, these modifications are not likely responsible for the pI shift. Measuring the extent of methyl esterification on PCNA and caPCNA determines malignant from non-malignant (caPCNA from nmPCNA). Using the methods disclosed herein, the methylesterification levels of caPCNA and nmPCNA are determined and compared for diagnosis of malignancy.

### Example 3

### Semi-Quantitation of Methyl Esterified caPCNA Peptides

The identification of methyl esters on caPCNA with respect to the pI of the isoform was further investigated. PCNA has a calculated pI of approximately 4.5 and the pI of caPCNA, as determined after calibration of the 2D-PAGE gel using the pIs of surrounding proteins, was slightly higher, approximately 4.6. In contrast, if 100% of all 16 acidic residues were methyl esterified, the protein's pI would likely shift basic more dramatically than 0.1 pH units (e.g., 5.66). There may be additional residues that are modified to produce the basic or nmPCNA isoform. The nmPCNA isoform may also be methyl esterified on different and/or additional residues than caPCNA. The methods disclosed herein enable one of ordinary skill in the art to determine methylesterification levels of caPCNA and nmPCNA. The relative abundances of the methyl esterified peptides was measured and compared to the unmodified peptides. This was accomplished by measuring and comparing the peak areas of each unmodified peptide and its methyl esterified counterpart. Comparison of the peak areas revealed a relative abundance for each methyl ester identified in this LC-MS/MS experiment. Each of the peptides show only partial methyl esterification (<25%) when the peak areas are compared. Therefore the caPCNA isoform is likely to be comprised of a heterogeneous population of PCNA molecules with the same pI. In other words, a single caPCNA molecule likely exhibits one or few methyl esters, but not 16. But the one or few methyl esters can occur on 16 different residues throughout the protein.

This heterogeneity of caPCNA is illustrated by the presence of methyl esterification on the C-terminal peptide of caPCNA. The unmodified peptide, IEDEEGS (SEQ ID NO: 17) (778 m/z), eluted at 28.9 min (FIG. 5A) and the CID spectrum of this peptide is consistent with a peptide containing unmodified acidic residues (FIG. 5B). Interestingly, in the selected ion chromatogram for methyl esterified species (792 m/z) of this peptide, two peaks are identified with 2-3 min increased retention times. This is likely due to an increased hydrophobic character and loss of charge imparted by methyl esterification. The resolution of these two peaks was therefore indicative of a difference in structure of these peptides. Inspection of the CID spectra identifies that the peptides are methyl esterified but on different residues (FIGS. 5C and 5D). Because no peptides harboring methyl esters on both residues were observed, the appearance of these peptides most likely resulted from the analysis of a heterogeneous population of caPCNA.

It is possible that the observed heterogeneity and low percentage of modified species could be the result of the facile lability of the methyl ester modifications themselves. Some reports indicate that protein methyl esterification modifications were short lived in neutral and basic solutions. Therefore, protein methyl esters, like those found on caPCNA, can spontaneously hydrolyze leaving an unmodified residue and methanol. Additionally, the basic and oxidizing conditions of SDS-PAGE can also lead to loss of methyl esters from PCNA, and attempts to resolve the basic PCNA isoform, a highly methyl esterified form of PCNA, to its basic pI appears to display a spontaneous regression towards a more acidic pI.

A high level of methyl esters likely cause PCNA to focus to a basic pI as shown in the immunoblot (approximately pH 8.8-9.0). However, focusing of this isoform may not be uniform (streaky) and may be present at a lower intensity. The basic pHs at which this isoform resolves may not be conducive to maintain all of the methyl esterification on the protein. The inability to focus at a specific pI observed on this gel is likely due to the concomitant loss of one ore more of the methyl esters due to the focusing at a basic pH. Spontaneous hydrolysis of the methyl esters occur, liberating methanol and an unmodified amino acid side chain. Regeneration of the acidic side chains by this “basic hydrolysis” likely causes PCNA's pI to shift from a basic one to a more acidic one, as evidenced by the accumulation of PCNA towards the mores acidic side of the gel (pH 7).

Identification and analysis of methylesterification can be performed under conditions that minimize loss of methylesters. For example, a method describing acidic 2D-PAGE that uses conditions able to preserve protein methyl esters has been described (O'Connor et al., Anal Biochem 1985, 148, 79-86). However, many of the available proteases that recognize PCNA are active in neutral to basic pHs and it is possible that some significant amount of methyl esterification would be lost during the digestion.

In the intact cell or in extracts, the enzyme(s) responsible for the methyl esterification may be active and can modify residues that have lost methyl esters to spontaneous hydrolysis. Separation of PCNA from the enzyme(s) responsible for the methyl esterification and incubation in conditions supporting hydrolysis (e.g., pH above 7.0) may lead to loss of one or more methyl esters. For example, such loss of methyl esters can be minimized by maintaining a slightly acidic condition during sample handling and analysis.

TABLE I
Methylesterification state of various caPCNA-derived peptides.

| | | | | | Methyl | |
|---|---|---|---|---|---|---|
| | Observed | Charge | Calc. | Peak | Ester | |
| Peptide Sequence^{a} | m/z | State | mass | Area | (%)^{b} | Score^{c} |
| MₒFEₘAR (SEQ ID NO: 1) | 343.37 | 2 | 682.31 | 5.9 × 10⁶ | 3.6 | 19 |
| IEₘDEEGS (SEQ ID NO: 2) | 792.30 | 1 | 791.32 | 2.8 × 10⁷ | 7.3 | 27 |
| IEDEEₘGS (SEQ ID NO: 3) | 792.47 | 1 | 791.32 | 2.7 × 10⁷ | 7.0 | 25 |
| VSDYEₘMₒK (SEQ ID NO: 4) | 451.9 | 2 | 900.39 | 8.1 × 10⁶ | 11.4 | 46 |
| MₒPSGEₘFAR (SEQ ID NO: 5) | 463.20 | 2 | 923.42 | 1.4 × 10⁷ | 2.5 | 50 |
| LSQTSNVDₘK (SEQ ID NO: 6) | 503.96 | 2 | 1004.51 | 1.5 × 10⁶ | 21.1 | 40 |
| C_{ca}AGNEₘDIITLR (SEQ ID NO: 7) | 639.44 | 2 | 1274.63 | 1.7 × 10⁷ | 8.8 | 66 |
| FSASGEₘLGNGNIK (SEQ ID NO: 8) | 655.11 | 2 | 1306.65 | 7.6 × 10⁷ | 2.7 | 99 |
| AEDNADTLALVFEAPNQEₘK (SEQ ID NO: 9) | 1045.93 | 2 | 2088.00 | 5.0 × 10⁷ | 3.5 | 97 |
| AEₘDNADTLALVFEAPNQEK(SEQ ID NO: 10) | 1046.01 | 2 | 2088.00 | 2.0 × 10⁷ | 3.9 | 89 |
| AEDₘNADTLALVFEAPNQEK(SEQ ID NO: 11) | 1045.31 | 2 | 2088.00 | 4.1 × 10⁷ | 12.7 | 102 |
| AEDNADTLALVFEₘAPNQEK(SEQ ID NO: 12) | 1045.69 | 2 | 2088.00 | 3.4 × 10⁷ | 4.5 | 94 |
| LMₒDₘLDVEQLGIPEQEYSC_{ca}VVK | 1249.50 | 2 | 2494.20 | 9.5 × 10⁷ | 10.3 | 78 |
| (SEQ ID NO: 13) | | | | | | |
| ATPLSSTVTLSMₒSADVPLVVEₘYK | 1220.83 | 2 | 2437.27 | 1.35 × 10⁷ | 8.3 | 95 |
| (SEQ ID NO: 14) | | | | | | |
| LSQTSNVDKEEEAVTIEMₒNEₘPVQLTFALR | 1108.28 | 3 | 3320.64 | 3.43 × 10^{9d} | 8.7 | 50 |
| (SEQ ID NO: 15) | | | | | | |

^{a}Peptide modifications presented are oxidized methionine (Mₒ), carbamidomethyl cysteine (C_{ca}), methyl esterified glutamic acid (Eₘ), and methyl esterified aspartic acid (Dₘ).
^{b}Percent methyl ester was calculated by dividing the peak areas of the methyl esterified peptides by the combined peak areas for the methyl esterified and unmodified peptides.
^{c}Mascot scores are reported as −10log(P), where P is the probability that the match is a random event.
^{d}Data generated using an LCQ DECA XP compared to an LCQ Advantage.

TABLE II
Amino acid positions of methylesters on caPCNA

| Methyl | | Position |
|---|---|---|
| Ester | Residue | (1-261 a.a.) |
| 1 | Glutamic acid | 3 |
| 2 | Glutamic acid | 85 |
| 3 | Glutamic acid | 93 |
| 4 | Aspartic Acid | 94 |
| 5 | Glutamic acid | 104 |
| 6 | Glutamic acid | 109 |
| 7 | Glutamic acid | 115 |
| 8 | Aspartic acid | 120 |
| 9 | Glutamic acid | 132 |
| 10 | Glutamic acid | 143 |
| 11 | Glutamic acid | 174 |
| 12 | Aspartic acid | 189 |
| 13 | Glutamic acid | 201 |
| 14 | Aspartic acid | 238 |
| 15 | Glutamic acid | 256 |
| 16 | Glutamic acid | 258 |

1. A purified methyltransferase, where said methyltransferase methylesterifies one or more acidic amino acid residues of proliferating cell nuclear antigen (PCNA); has a molecular weight of approximately 50 kDa; and has a secondary structure comprising 9 α-helices in its N-terminus and seven a-helices and nine β-sheets in its C-terminus.

2. The methyltransferase of claim 1 wherein said methyltransferase methylesterifies one or more glutamic acid residues that correspond to the amino acid positions 3, 85, 93, 94, 104, 109, 115, 120, 132, 143, 174, 189, 201, 238, 256, and 258 of SEQ ID NO: 37.

3. The methyltransferase of claim 1 wherein said methyltransferase is more than 95% pure.

4. The methyltransferase of claim 1 that when exposed to the peptide of SEQ ID NO: 33 in the presence of S-adenosyl-L-methionine produces a peptide comprising one or more of the following sequences
(SEQ ID NO: 1)
MFEₘAR;
(SEQ ID NO: 2)
IEₘDEEGS;
(SEQ ID NO: 3)
IEDEEₘGS;
(SEQ ID NO: 4)
VSDYEₘMK;
(SEQ ID NO: 5)
MPSGEₘFAR;
(SEQ ID NO: 6)
LSQTSNVDₘK;
(SEQ ID NO: 7)
CAGNEₘDIITLR;
(SEQ ID NO: 8)
FSASGEₘLGNGNIK;
(SEQ ID NO: 9)
AEDNADTLALVFEAPNQEₘK;
(SEQ ID NO: 10)
AEₘDNADTLALVFEAPNQEK;
(SEQ ID NO: 11)
AEDₘNADTLALVFEAPNQEK;
(SEQ ID NO: 12)
AEDNADTLALVFEₘAPNQEK;
(SEQ ID NO: 13)
LMDₘLDVDQLGIPEQEYSCVVK;
(SEQ ID NO: 14)
ATPLSSTVTLSMSADVPLVVEₘYK;
(SEQ ID NO: 15)
LSQTSNVDKEEEAVTIEMNEₘPVQLTFALR;
and
(SEQ ID NO: 31)
LMDLDVEQLGIPEQEₘYSCVVK

5. The methyltransferase of claim 1 comprising the sequence of SEQ ID NO: 33 or an amino acid sequence that is greater than 90% identical to the corresponding sequence of SEQ ID NO: 33.

6. The methyltransferase of claim 4 wherein amino acids 245-257 of said methyltransferase are identical to SEQ ID NO: 33.

7. A kit comprising the purified methyltransferase of claim 1; and proliferating cell nuclear antigen (PCNA) polypeptide or a peptide comprising an amino acid sequence selected from the group consisting of
(SEQ ID NO: 16)
MFEAR;
(SEQ ID NO: 17)
IEDEEGS;
(SEQ ID NO: 18)
IEDEEGS;
(SEQ ID NO: 19)
VSDYEMK;
(SEQ ID NO: 20)
MPSGEFAR;
(SEQ ID NO: 21)
LSQTSNVDK;
(SEQ ID NO: 22)
CAGNEDIITLR;
(SEQ ID NO: 23)
FSASGELGNGNIK;
(SEQ ID NO: 24)
AEDNADTLALVFEAPNQEK;
(SEQ ID NO: 25)
AEDNADTLALVFEAPNQEK;
(SEQ ID NO: 26)
AEDNADTLALVFEAPNQEK;
(SEQ ID NO: 27)
AEDNADTLALVFEAPNQEK;
(SEQ ID NO: 28)
LMDLDVEQLGIPEQEYSCVVK;
(SEQ ID NO: 29)
ATPLSSTVTLSMSADVPLVVEYK;
(SEQ ID NO: 30)
LSQTSNVDKEEEAVTIEMNEPVQLTFALR;
and
(SEQ ID NO: 32)
LMDLDVEQLGIPEQEYSCVVK.

8. The kit of claim 7 further comprising S-adenosyl-L-methionine.

9. A method for diagnosing cancer, said method comprising
obtaining a biological test sample from a patient;
conducting an assay for proliferating cell nuclear antigen (PCNA) targeted methyltransferase activity; and
measuring the amount of proliferating cell nuclear antigen (PCNA) that has been methylesterified by said biological test sample relative to a standard value.

10. The method of claim 9 wherein the standard value is established based on biological samples recovered from healthy patients.

11. The method of claim 9 wherein the standard value represents the amount of proliferating cell nuclear antigen (PCNA) that has been methylesterified by a second biological sample recovered from healthy tissues of the same patient.

12. The method of claim 9 wherein the biological test sample is a cell extract of a biopsy tissue sample.

13. (canceled)


SEQUENCE LISTING (45 sequences)

SEQ ID NO: 1 - protein, 5 aa, Artificial Sequence
  Description of Artificial Sequence Synthetic peptide
MFEAR

SEQ ID NO: 2 - protein, 7 aa, Artificial Sequence
  Description of Artificial Sequence Synthetic peptide
IEDEEGS

SEQ ID NO: 3 - protein, 7 aa, Artificial Sequence
  Description of Artificial Sequence Synthetic peptide
IEDEEGS

SEQ ID NO: 4 - protein, 7 aa, Artificial Sequence
  Description of Artificial Sequence Synthetic peptide
VSDYEMK

SEQ ID NO: 5 - protein, 8 aa, Artificial Sequence
  Description of Artificial Sequence Synthetic peptide
MPSGEFAR

SEQ ID NO: 6 - protein, 9 aa, Artificial Sequence
  Description of Artificial Sequence Synthetic peptide
LSQTSNVDK

SEQ ID NO: 7 - protein, 11 aa, Artificial Sequence
  Description of Artificial Sequence Synthetic peptide
CAGNEDIITLR

SEQ ID NO: 8 - protein, 13 aa, Artificial Sequence
  Description of Artificial Sequence Synthetic peptide
FSASGELGNGNIK

SEQ ID NO: 9 - protein, 19 aa, Artificial Sequence
  Description of Artificial Sequence Synthetic peptide
AEDNADTLALVFEAPNQEK

SEQ ID NO: 10 - protein, 19 aa, Artificial Sequence
  Description of Artificial Sequence Synthetic peptide
AEDNADTLALVFEAPNQEK

SEQ ID NO: 11 - protein, 19 aa, Artificial Sequence
  Description of Artificial Sequence Synthetic peptide
AEDNADTLALVFEAPNQEK

SEQ ID NO: 12 - protein, 19 aa, Artificial Sequence
  Description of Artificial Sequence Synthetic peptide
AEDNADTLALVFEAPNQEK

SEQ ID NO: 13 - protein, 21 aa, Artificial Sequence
  Description of Artificial Sequence Synthetic peptide
LMDLDVEQLGIPEQEYSCVVK

SEQ ID NO: 14 - protein, 23 aa, Artificial Sequence
  Description of Artificial Sequence Synthetic peptide
ATPLSSTVTLSMSADVPLVVEYK

SEQ ID NO: 15 - protein, 29 aa, Artificial Sequence
  Description of Artificial Sequence Synthetic peptide
LSQTSNVDKEEEAVTIEMNEPVQLTFALR

SEQ ID NO: 16 - protein, 5 aa, Artificial Sequence
  Description of Artificial Sequence Synthetic peptide
MFEAR

SEQ ID NO: 17 - protein, 7 aa, Artificial Sequence
  Description of Artificial Sequence Synthetic peptide
IEDEEGS

SEQ ID NO: 18 - protein, 7 aa, Artificial Sequence
  Description of Artificial Sequence Synthetic peptide
IEDEEGS

SEQ ID NO: 19 - protein, 7 aa, Artificial Sequence
  Description of Artificial Sequence Synthetic peptide
VSDYEMK

SEQ ID NO: 20 - protein, 8 aa, Artificial Sequence
  Description of Artificial Sequence Synthetic peptide
MPSGEFAR

SEQ ID NO: 21 - protein, 9 aa, Artificial Sequence
  Description of Artificial Sequence Synthetic peptide
LSQTSNVDK

SEQ ID NO: 22 - protein, 11 aa, Artificial Sequence
  Description of Artificial Sequence Synthetic peptide
CAGNEDIITLR

SEQ ID NO: 23 - protein, 13 aa, Artificial Sequence
  Description of Artificial Sequence Synthetic peptide
FSASGELGNGNIK

SEQ ID NO: 24 - protein, 19 aa, Artificial Sequence
  Description of Artificial Sequence Synthetic peptide
AEDNADTLALVFEAPNQEK

SEQ ID NO: 25 - protein, 19 aa, Artificial Sequence
  Description of Artificial Sequence Synthetic peptide
AEDNADTLALVFEAPNQEK

SEQ ID NO: 26 - protein, 19 aa, Artificial Sequence
  Description of Artificial Sequence Synthetic peptide
AEDNADTLALVFEAPNQEK

SEQ ID NO: 27 - protein, 19 aa, Artificial Sequence
  Description of Artificial Sequence Synthetic peptide
AEDNADTLALVFEAPNQEK

SEQ ID NO: 28 - protein, 21 aa, Artificial Sequence
  Description of Artificial Sequence Synthetic peptide
LMDLDVEQLGIPEQEYSCVVK

SEQ ID NO: 29 - protein, 23 aa, Artificial Sequence
  Description of Artificial Sequence Synthetic peptide
ATPLSSTVTLSMSADVPLVVEYK

SEQ ID NO: 30 - protein, 29 aa, Artificial Sequence
  Description of Artificial Sequence Synthetic peptide
LSQTSNVDKEEEAVTIEMNEPVQLTFALR

SEQ ID NO: 31 - protein, 21 aa, Artificial Sequence
  Description of Artificial Sequence  Synthetic peptide
LMDLDVEQLGIPEQEYSCVVK

SEQ ID NO: 32 - protein, 21 aa, Artificial Sequence
  Description of Artificial Sequence  Synthetic peptide
LMDLDVEQLGIPEQEYSCVVK

SEQ ID NO: 33 - protein, 441 aa, Homo sapiens
MAVVPASLSGQDVGSFAYLTIKDRIPQILTKVIDTLHRHKSEFFEKHGEEGVEAEKKAISLLSKLRNELQTDKPFIPLVEKFVDTDIWNQYLEYQQSLLNESDGKSRWFYSPWLLVECYMYRRIHEAIIQSPPIDYFDVFKESKEQNFYGSQESIIALCTHLQQLIRTIEDLDENQLKDEFFKLLQISLWGNKCDLSLSGGESSSQNTNVLNSLEDLKPFILLNDMEHLWSLLSNCKKTREKASATRVYIVLDNSGFELVTDLILADFLLSSELATEVHFYGKTIPWFVSDTTIHDFNWLIEQVKHSNHKWMSKCGADWEEYIKMGKWVYHNHIFWTLPHEYCAMPQVAPDLYAELQKAHLILFKGDLNYRKLTGDRKWEFSVPFHQALNGFHPAPLCTIRTLKAEIQVGLQPGQGEQLLASEPSWWTTGKYGIFQYDGPL

SEQ ID NO: 34 - protein, 286 aa, Escherichia coli
MTSSLPCGQTSLLLQMTERLALSDAHFRRISQLIYQRAGIVLADHKRDMVYNRLVRRLRSLGLTDFGHYLNLLESNQHSGEWQAFINSLTTNLTAFFREAHHFPLLADHARRRSGEYRVWSAAASTGEEPYSIAMTLADTLGTAPGRWKVFASDIDTEVLEKARSGIYRHEELKNLTPQQLQRYFMRGTGPHEGLVRVRQELANYVDFAPLNLLAKQYTVPGPFDAIFCRNVMIYFDQTTQQEILRRFVPLLKPDGLLFAGHSENFSHLERRFTLRGQTVYALSKD

SEQ ID NO: 35 - protein, 227 aa, Homo sapiens [sequence omitted]

SEQ ID NO: 36 - DNA, 1326 bp, Homo sapiens
atggctgtcgtcccggcgtctctctcaggacaggacgtgggatcatttgcatatcttacaattaaagacagaataccacagatcttaactaaggttattgatacattgcatcgacataaaagtgaattttttgagaaacacggagaggaaggcgtggaagctgaaaagaaagctatctctctcctttctaaattacggaatgaattgcaaacagataaaccatttatccccttggttgagaaatttgttgatactgatatatggaatcagtacctagaatatcaacagagtcttttaaatgaaagtgatggaaaatcaagatggttctactcaccgtggttgttggtagaatgttacatgtatcgaagaattcatgaagcaattatccagagtccaccaatcgattactttgatgtatttaaagaatcaaaagagcaaaatttctatgggtcacaggaatccatcattgctttatgtactcacctgcaacaattgataagaactattgaagacctagatgaaaatcagctgaaagatgagttttttaaacttctgcagatttcactgtggggaaataagtgtgatctgtctctctcaggtggagaaagtagttctcagaataccaatgtactaaattcattggaagacctaaaacctttcattttattgaatgatatggaacatctttggtcattgcttagcaattgcaagaaaacaagagaaaaagcttctgctactagagtgtatattgttctcgataattctggatttgagcttgttacagatttaatattagccgacttcttgttgtcctctgaactggctactgaggttcatttttatggaaaaacaattccatggtttgtttctgatactactatacatgattttaattggttaattgaacaggtaaaacacagtaatcataagtggatgtccaagtgtggggctgactgggaagagtatattaaaatgggtaaatgggtttaccacaatcatatattttggactctgcctcatgagtactgtgcaatgcctcaggttgcacctgacttatatgctgaactacagaaggcacatttaattttattcaagggtgatttgaattacaggaagttgacaggtgacagaaaatgggagttttctgttccatttcatcaggctctgaatggcttccatcctgcaccactctgtaccataagaacattaaaagctgaaattcaggttggtctgcagcctgggcaaggggaacagctcctggcctctgagcccagctggtggaccactggaaaatatggaatatttcagtacgatggtcccctttga

SEQ ID NO: 37 - protein, 261 aa, Artificial Sequence
  Description of Artificial Sequence Synthetic peptide
MFEARLVQGSILKKVLEALKDLINEACWDISSSGVNLQSMDSSHVSLVQLTLRSEGFDTYRCDRNLAMGVNLTSMSKILKCAGNEDIITLRAEDNADTLALVFEAPNQEKVSDYEMKLMDLDVEQLGIPEQEYSCVVKMPSGEFARICRDLSHIGDAVVISCAKDGVKFSASGELGNGNIKLSQTSNVDKEEEAVTIEMNEPVQLTFALRYLNFFTKATPLSSTVTLSMSADVPLVVEYKIADMGHLKYYLAPKIEDEEGS

SEQ ID NO: 38 - protein, 198 aa, Bos taurus
MAGPPASLSARDVGSFAYLSVKDRSPQILTKAIDTLHRHKSEFFEKHGEKGLEAEKKAISLLSKLRNELQTDKPIVPLVEKFVDTDIWNQYLEYQQSLLNESDGKPRWFLSPWLFVECYMYRRIHEAIIQSPPIDDFDIFKEFKDQNFFESQESIIALCTHLQELRKTIEDLDENQLKNEFFKVLQISLWGNKCDLSL

SEQ ID NO: 39 - protein, 197 aa, Mus musculus
MAESPAFLSAKDEGSFAYLTIKDRTPQILTKVIDTLHRHKSEFFEKHGEEGIEAEKKAISLLSKLRNELQTDKPITPLVDKCVDTHIWNQYLEYQRSLLNEGDGEPRWFFSPWLFVECYMYRRIHEAIMQSPPIHDFDVFKESKEENFFESQGSIDALCSHLLQLKPVKGLREEQIQDEFFKLLQISLWGNKCDLSL

SEQ ID NO: 40 - protein, 201 aa, Danio rerio
MEAEGMLPPQSLSAKFEGSFAYLTVRDRLPTILTKVVDTLHRNKDNFYKEYGEEGTEAEKRAISFLSRLRNELQTDKPVLALTDNAEDTQAWNEYMERQQDLMENGQLVSWFKSPWLYVECYMYRRIQEALYMNPPMHNFDPFKEGKTQSYFESQQAIKYLCTYLQELITNMENMTEIQLRENFLKLIQVSLWGNKCDLSI

SEQ ID NO: 41 - protein, 199 aa, Drosophila melanogaster
MGSETDFDAKNGIVDGPTPPHTELAALYKQSFAYYTFRVRLPSTLATIADSLVKDKDVLLATYGAAAEADIEQTTKEVRQLRDDILSNGPLLPFGENDSDSEVWNAFLEKLPKEKRTYFSVCWLYAECYMYRKISSIFRATAHLAAYDYFSQQKQTATKLSVDAMLAVAKATRHNERNSDTFRQLIKLNLWGNRCDLSI

SEQ ID NO: 42 - protein, 202 aa, Caenorhabditis elegans
MENADEYDHLAPKLRGKKEGTFAYYTVRDRWPKIVTGLVDQLAQKRASLIEKYGSEVESDIAAILEVFSKLRYEIMTDKPLCNLMDTQLDSEMWRNLLSDMRTAAMPDEVEDLTFFKGPWLFVECWLYRFIWSTFAKTIRLSEYDYFQDSKRKNFLDHLPQIEESAAFINKISAKDAPVHELFGINTILKMSLWGNRADMSL

SEQ ID NO: 43 - protein, 202 aa, Schizosaccharomyces pombe
MGLKLLHPPKPYAMTSDPESYASVCVMKKWPIIATNVIDEVSRNISKALEAGMSDKAAYVTQGKEIISLLNQLKYDLQHNRPLKPLVGQGPDIDDYNEELEQVGPLTWGDAPWLYAGCYFYRIMSLFFQARSEWNRHDPFFEQKDFTLRSSKSAIEEFAKRYVHLNSELASIQENKDDKAAYMIFVEMAEISLWGNAIDLGL

SEQ ID NO: 44 - protein, 198 aa, Saccharomyces cerevisiae
MTIPGRFMTIDKGTFGEYTASTRWPIIIQNAIDDLSKHQETEKSNGTKFEQGEVIKKELKEFRQEIIDRVPLRPFTEEEIKIANVPLSFNEYLKKHPEVNWGAVEWLFSEVYLYRRVNVLFQRQCEWAKFDIFNRLKQSTFESSFYGVVELALRYENLLPQLREMKQNPGNEIDDILKVLFKEFIEISLWGNATDLSL

SEQ ID NO: 45 - protein, 261 aa, Artificial Sequence; identical to SEQ ID NO: 37
  Description of Artificial Sequence Synthetic peptide